(19) United States
(12) Patent Application Publication
(10) Pub. No.: US 2011/0311457 A1
(43) Pub. Date: Dec. 22, 2011

(54) METHODS FOR IMAGING VASCULAR INFLAMMATION USING IMPROVED NANOPARTICLE CONTRAST AGENTS

(75) Inventors: H. Donlon Skerrett, Wayne, PA (US); Joseph Patterson, Lansdale, PA (US); Robert W. Lee, Boyertown, PA (US); Zahi A. Fayad, Larchmont, NY (US)

(73) Assignee: NANOSCAN IMAGING, LLC, Lansdale, PA (US)

(21) Appl. No.: 12/670,756

(22) PCT Filed: Jul. 28, 2008
(86) PCT No.: PCT/US08/71391

§ 371 Date: Jan. 26, 2010

Related U.S. Application Data
(60) Provisional application No. 60/962,117, filed on Jul. 26, 2007.

(51) Int. Cl. A61K 49/04; B32B 5/16; B82Y 5/00

(52) U.S. Cl. 424/9.45; 428/402; 977/773; 977/788; 977/928

(57) ABSTRACT

The present invention also provides methods and compositions for imaging and evaluating, e.g., blood flow or inflammation in a subject. Such evaluations are important in a number of clinical diagnoses, including assessing organ damage associated with angina pectoris, heart attack, stroke, cancer, atherosclerosis, and the like, as well as assessing vessel leakages associated with aneurisms, diffuse bleedings after trauma, and the like.

### RELATED APPLICATION

This application claims the benefit of U.S. Provisional Application Ser. No. 60/962,117, filed Jul. 26, 2007, the contents of which are hereby incorporated by reference.

### BACKGROUND OF THE INVENTION

Contrast agents are injected into the vasculature of a subject in order to image blood flow in veins and arteries. Many of the agents currently used for imaging are water soluble. These agents provide contrast in the lumen of the vessel so that a positive image of the lumen can be made and the path of blood flow in the vessel can be detected. Images are made shortly after administration, before the agent is cleared from the lumen. Images made using such methods provide information regarding, for example, vessel occlusion, the presence of aneurisms, neovasculariztion, and blood flow.

Different images can be made using nanoparticulate contrast agents. Certain of these agents can be taken up by phagocytic cells and, therefore, are useful in imaging sites of inflammation, e.g., the sites where phagocytic cells accumulate. For example, such agents can be used to image vascular plaque. Because these agents image phagocytes in vessel walls, they are useful in imaging ‘vulnerable’ or ‘active’ plaque (which may not be visible using standard imaging methods as it is often not occlusive). Vulnerable plaque has the tendency to break away from the vessel and, if loosened, can travel through the vascular system causing a coronary attack, a stroke if in the region of the brain, or an occlusion of a vessel if in the leg. It has been shown that plaque composition rather than the severity of a stenosis more accurately predicts the risk of plaque rupture and acute clinical complications of coronary artery disease.

While conventional imaging and detection using intravenous contrast medium enhancement is currently available, these methods and media are dependent on many complex factors, including the type of media, volume, concentration, injection technique, catheter size and site, imaging technique, cardiac output and tissue characteristics. Only some of these factors are controllable by radiologists (see, e.g., Bae, K. T., Heikin, J. P. and Brink, J. A. (1998) Radiology 207:647-655 and Bae, K. T., Heikin, J. P. and Brink, J. A. (1998) Radiology 207:657-662). For example, mixing or streak artifacts can compromise interpretation of computed tomography (CT) scans of the abdomen. These artifacts are primarily related to the first pass (arterial phase) effects of intravenous contrast on vascular enhancement (see, e.g., Silverman, P. M. et al. (1995) Radiographics 15:25-36 and Herts, B. R., Einstein, D. M. and Paushter, D. M. (1993) J. Roentgenol. 161:1185-1190). Diffusion of contrast media outside the vascular space not only degrades lesion conspicuity, but also requires that imaging be formed within two minutes after the start of injection. Very rapid elimination through the kidneys renders these substances less suitable for imaging of the vascular system since the imaging window may be too short for obtaining acceptable contrast. All of these difficulties are accentuated in indications that require a consistent contrast enhancement of the vascular blood pool in various vascular beds.

One nanoparticulate contrast agent which has been used to image vascular plaque, N1177, was designed to be taken up by phagocytic cells when administered by the subcutaneous route in order to assess regional lymph nodes. However, the composition was not optimized for intravenous administration. In addition, the composition was not optimized for assessment of intravascular inflammation. Improved contrast agents for vascular imaging would be of great benefit.

### SUMMARY OF THE INVENTION

The present invention provides, at least in part, compositions and methods for vascular imaging which are optimized for intravenous administration. In one embodiment, such compositions are optimized to image intravascular inflammation. Prior to the instant invention, it was not clear whether changes to one or more physical (e.g., size, size distribution and shape) or chemical properties (e.g., adsorbed surfactants and excipients) of a nanoparticulate contrast agent would optimize its imaging capabilities when administered intraveneously, e.g., its ability to image intravascular inflammation. The instant invention is based, at least in part, on the finding that compositions comprising nanoparticulate contrast agents having a mean particle size of less than about 150 nm on a per weight or per volume basis are optimal for vascular imaging as well as for detection of intravenous inflammation. The use of such particles increases the half-life of the particles in the circulation and limits uptake of the particles by the macrophages of the reticuloendothelial system. In addition, in one embodiment, it is possible to use a lower dose of such particles and still obtain an image useful in obtaining a diagnosis, e.g., an image of macrophages in the vessel wall of a subject suffering from vascular inflammation. Accordingly, in one embodiment, the present invention is directed to compositions and methods for imaging, detecting, or evaluating the condition of the vasculature in a subject. In one embodiment, inflammation is detected by visualizing accumulated phagocytes, e.g., activated macrophages at sites of intravascular inflammation, e.g., in vascular plaque, in particular vulnerable plaque.

In one aspect, the invention pertains to a composition comprising a crystalline iodinated nanoparticle contrast agent having a mean particle size of about 100 nanometers to about 150 nanometers.

In another embodiment, the invention pertains to a composition comprising a crystalline iodinated nanoparticle contrast agent having a particle size distribution of between about 80 and about 350 nanometers in diameter as measured by asymmetrical flow field fractionation.

In yet another embodiment, the invention pertains to a composition comprising a crystalline iodinated nanoparticle contrast agent having a particle size distribution of between about 20 and about 120 nanometers in diameter as measured by photon correlation spectroscopy (PCS).

In another embodiment, the invention pertains to a composition comprising a crystalline iodinated nanoparticle contrast agent having a particle size distribution in which 100% of particles are less than about 200 nanometers as measured by X-ray disc centrifuge sedimentometry (XDC).

In one embodiment, the contrast agent is an ester of diatrizoic acid. In one embodiment, contrast agent comprises iodine. In one embodiment, the contrast agent is 6-ethoxy-6-oxohexy-3,5-bis(acetylamino)-2,4,6-triiodobenzoate

In one embodiment, the invention pertains to an in vivo method for obtaining an image of accumulated macrophages in a blood vessel of a subject comprising:

a) administering an effective amount of a composition comprising a nanoparticulate contrast agent having mean diameter of less than or equal to about 150 nanometers to the subject intravenously; and

b) detecting the contrast agent.

In one aspect, the invention pertains to an in vivo method for obtaining an image of plaque accumulation in a blood vessel of a subject comprising:

a) administering an effective amount of a composition comprising a nanoparticulate contrast agent having a mean diameter of about 150 nanometers or less the subject intravenously; and

b) waiting a time sufficient after administration of the contrast agent to allow the contrast agent to be taken up by macrophages in vascular plaque that may be present in the subject; and

c) detecting the contrast agent taken up by the macrophages thereby obtaining an image of vascular plaque that may be present in the subject.

In another aspect, the invention pertains to an in vivo method for predicting risk of vascular disease by obtaining and evaluating an image of accumulated macrophages within a blood vessel of a subject comprising:

a) administering to the subject an effective amount of a composition comprising a nanoparticulate contrast agent having a mean diameter of about 150 nanometers or less;

b) waiting a time sufficient after administration of the contrast agent to allow the contrast agent to be taken up by macrophages in vascular plaque that may be present in the subject; and

c) detecting the contrast agent taken up by the macrophages thereby obtaining an image of accumulated macrophages that may be present in the subject.

d) predicting risk of vascular disease in the subject based on the image formed.

In one embodiment, the prediction is made based on a quantitative measure of the accumulation of the contrast agent in the macrophages in the vessel wall of the subject.

In another embodiment, the vascular disease is selected from the group consisting of atherosclerosis, coronary artery disease (CAD), myocardial infarction (MI), ischemia, stroke, peripheral vascular diseases, and venous thromboembolism.

In yet another aspect, the invention pertains to a method for diagnosing atherosclerosis in a human subject, comprising

a) examining an image for the presence or absence of vascular plaque, wherein the image is obtained by:
i. administering to a human subject at risk for developing vascular plaque an effective amount of a composition comprising the nanoparticulate contrast agent 6-ethoxy-6-oxohexy-3,5-bis(acetylamino)-2,4,6-triiodobenzoate having a mean diameter of about 150 nanometers or less;
ii. waiting a time sufficient after administration of the contrast agent to allow the contrast agent to be taken up by macrophages and for the amount of the contrast agent in the lumen of the vessel to be imaged to be reduced to an amount which allows macrophages in vascular plaque that may be present in the human subject to be visualized; and
iii. detecting the contrast agent taken up by the macrophages, and

b) concluding whether vulnerable plaque is present in the image, wherein the presence of vascular plaque is indicative of atherosclerosis, to thereby diagnose atherosclerosis in the human subject.

In one aspect, the invention pertains to an in vivo method for obtaining an image of vulnerable vascular plaque that may be present in a subject at risk for developing vascular plaque, comprising

a) administering to a subject at risk for developing vascular plaque or known to have a vascular plaque an effective amount of a composition comprising a nanoparticulate contrast agent having a mean diameter of about 150 nanometers or less;

b) waiting a time sufficient after administration of the contrast agent to allow the contrast agent to be taken up by macrophages in vulnerable vascular plaque that may be present in the subject; and

c) constructing an image from data obtained by detecting the contrast agent taken up by the macrophages to thereby obtaining an image of vulnerable vascular plaque that may be present in the subject.

In another aspect, the invention pertains to an in vivo method for obtaining an image of a vascular blood pool in a subject comprising:

a) administering an effective amount of a composition comprising a nanoparticulate contrast agent having a mean diameter of about 150 nanometers or less to the subject intravenously; and

b) detecting the contrast agent present in a blood vessel of the subject to thereby obtain an image of a vascular blood pool in a subject.

In one embodiment, the vascular blood pool is chosen from the group consisting of a liver blood pool, a pancreatic blood pool, a lung blood pool, a cardiac blood pool, a splenic blood pool, and a brain blood pool.

In one embodiment, the vascular blood pool is a cardiac blood pool. In one embodiment, the vascular blood pool is a splenic blood pool. In one embodiment, the vascular blood pool is a pancreatic blood pool. In one embodiment, the vascular blood pool is a lung blood pool. In one embodiment, the vascular blood pool is a brain blood pool.

In another aspect, the invention pertains to an in vivo method for obtaining an image of phagocytic cells in the brain of a subject comprising:

a) administering an effective amount of a composition comprising a nanoparticulate contrast agent having a mean diameter of about 150 nanometers or less to the subject intravenously; and

b) waiting a time sufficient after administration of the contrast agent to allow the contrast agent to be taken up by phagocytic cells that may be present in the brain of the subject; and

c) detecting the contrast agent taken up by the phagocytic cells thereby obtaining an image of phagocytic cells that may be present in the brain of the subject.

In one embodiment, the phagocytic cells are associated with neural plaques.

In one embodiment, the invention pertains to an in vivo method for detecting the presence of a tumor that may be present at a site of interest in a subject comprising:

a) administering an effective amount of a composition comprising a nanoparticulate contrast agent having a mean diameter of about 150 nanometers or less to the subject intravenously such that it is present in the vasculature of the subject; and

b) detecting the contrast agent present in the vasculature at the site of interest,

to thereby obtain an image of the vasculature associated with a tumor that may be present at a site of interest in the subject.

In one embodiment, the image is evaluated for areas of increased formation of blood vessels or leakage of contrast agent from blood vessels.

In another aspect, the invention pertains to in vivo method for obtaining an image of phagocytic cells at a site of interest in a subject comprising:

a) administering an effective amount of a composition comprising a nanoparticulate contrast agent having a mean diameter of about 150 nanometers or less to the subject intravenously; and

b) waiting a time sufficient after administration of the contrast agent to allow the contrast agent to be taken up by phagocytic cells that may be present at the site of interest in the subject; and

c) detecting the contrast agent taken up by the phagocytic cells thereby obtaining an image of phagocytic cells that may be present at the site of interest in the subject.

In one embodiment, the phagocytic cells are present at the site of a tumor.

In one embodiment, the composition a particle size distribution in which 100% of the contrast agent has a particle size of not more than 400 nanometers.

In one embodiment, the contrast agent has a mean particle size of between about 100 and 150 nm.

In one embodiment, the contrast agent has a particle size distribution of between about 80 and about 350 nanometers in diameter as measured by asymmetrical flow field fractionation.

In one embodiment, the contrast agent has a particle size distribution of between about 20 and about 120 nanometers in diameter as measured by photon correlation spectroscopy (PCS).

In one embodiment, the contrast agent has a mean particle size of between about 100 and 150 nm and a particle size distribution of between about 80 and about 350 nanometers in diameter as measured by asymmetrical flow field fractionation or of between about 20 and about 120 nanometers in diameter as measured by photon correlation spectroscopy (PCS).

In one embodiment, the method of detecting is selected from the group consisting of x-ray imaging, computed tomography (CT), computed tomography angiography (CTA), multi-detector CT (MDCT), electron beam (EBT), magnetic resonance imaging (MRI), magnetic resonance angiography (MRA), and positron emission tomography.

### BRIEF DESCRIPTION OF THE DRAWINGS

FIG. 1 shows imaging of macrophages in atherosclerotic plaques. Panel A shows axial views of the same atherosclerotic plaque in the aorta of a rabbit on computed tomography before, during and 2 hours after the injection of N1177 or the non specific contrast agent. Inserts are magnifications of the regions surrounding the aorta. The same window level and width were used for all axial views. Bar width, 2 cm. Panel B shows signal intensities on CT after the injection of N1177 or the conventional iodinated contrast agent in the aortic wall of atherosclerotic or control rabbits. HU, Hounsfield units.

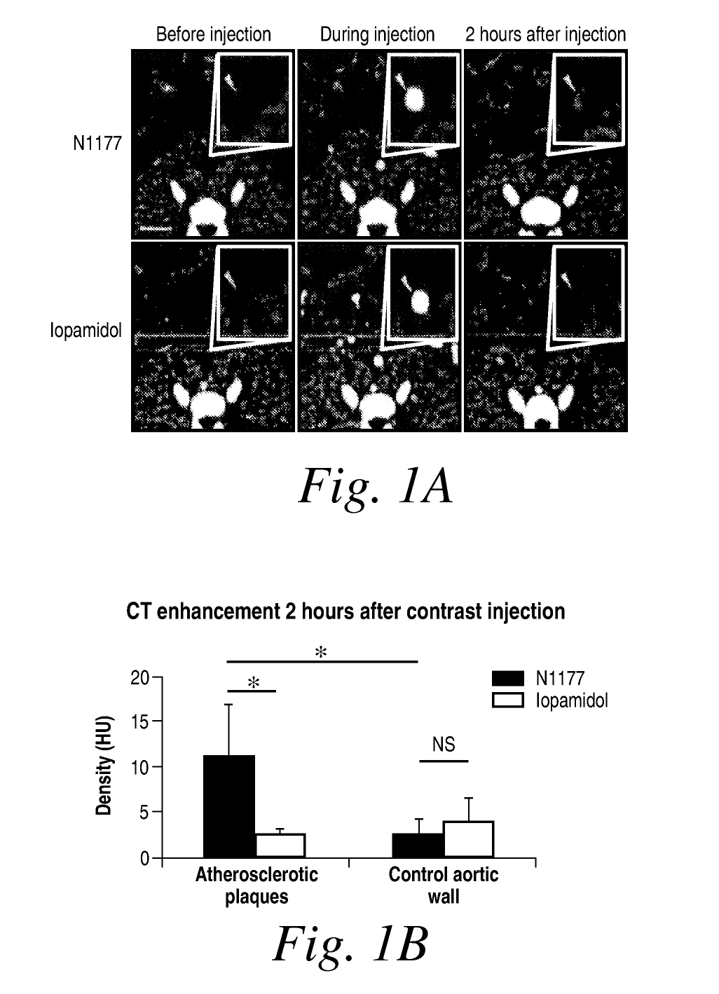

FIG. 2 shows Iodine versus time curves of blood samples obtained over a 24 hour time interval post injection.

FIG. 3 shows the percentage of agent found in the liver, spleen, lung, and kidney obtained for a large particle size (GLP; mean particle size 269.3, referred to herein as compound A) and small particle size (FID; mean particle size 128.5, referred to herein as compound B) formulation 24 hours after the administration of a 6-ml bolus. The results suggest that the large GLP batch has greater RES uptake (as observed by significant increases in the % injected dose (ID) of liver and spleen), relative to the smaller FID batch. Less than 4% of the injected dose was found in the kidneys 24 hours post injection for both formulations tested. No significant iodine was present in the heart. In the aorta 0.039% and 0.044% of the injected dose was obtained following administration of the large GLP and small FID batches, respectively. The percent ID was determined based upon the amount of iodine present in the tissue (mg) and the total amount of iodine administered.

FIG. 4 depicts the particle size distributions for compound A and B.

FIG. 5 shows plasma histamine levels after intravenous injection of compound B in comparison to Ultravist and NaCl in rats.

FIG. 6 shows the Plasmakinetics of compound A (panel A) and compound B (panel B)

FIG. 7 shows measurements of AHU in various tissues. FIG. 7A depicts measurements of AHU: kinetic in liver parenchyma after i.v. application. Compound A and Compound B show a vessel opacification in the liver parenchyma as compared to ultravist; FIG. 7B depicts measurements of AHU: kinetic in renal cortex after i.v. application; FIG. 7C depicts measurements of AHU: kinetic in aorta after i.v. application. Compound A and Compound B show a vessel opacification in the aorta as compared to ultravist; FIG. 7D depicts measurements of AHU: kinetic in vena cava after i.v. application. Compound A and Compound B show a vessel opacification in the vena cava as compared to ultravist

FIG. 8 shows the mapping of tumor perfusion using different compounds. FIG. 8A depicts mapping of tumor perfusion after Ultravist injection; FIG. 8B depicts mapping of tumor perfusion after compound B injection

### DETAILED DESCRIPTION OF THE INVENTION

The present invention provides optimized compositions and methods for imaging, detecting, and evaluating the vasculature of a subject, e.g., for detecting and evaluating vascular inflammation. The imaging of intravascular inflammation is important, for example, for the prediction and/or diagnosis of localized and generalized diseases and disorders and/or organ, tissue, or vessel damage (e.g., ischemic, inflamed, injured, infected, or healing organs, tissues, or vessels, vascular wall damage, peripheral vascular disease, and the like). The invention is not limited to the particular vascular tissue, vascular beds or organ tissues imaged.

The present invention is also directed to compositions and methods for imaging, detecting, and evaluating phagocytes, e.g., activated macrophages, such as are found at sites of intravascular inflammation in the body, including, e.g., vulnerable plaque. Vulnerable plaque contains macrophages, e.g., activated macrophages, which accumulate in arterial walls. The contrast agents of the invention are taken up by macrophages, e.g., activated macrophages.

Visualization of macrophages that have taken up the agent at sites of inflammation is possible using routine imaging technology, e.g., by x-ray imaging, ultrasonagraphy, computed tomography (CT), computed tomography angiography (CTA), multidetector-row CT (MDCT), electron beam (EBT), magnetic resonance imaging (MRI), magnetic resonance angiography (MRA), positron emission tomography, and other imaging technologies.

Preferably, images of sites of potential vascular inflammation are made after a sufficient amount of the contrast agent has left the vascular space such that a positive image of macrophages that have taken up the agent rather than a positive image of the lumen of vessel is made (e.g., the image is made post-lumenal contrast). In one embodiment, an image of a vessel is made after waiting a time sufficient after administration of the contrast agent to allow the contrast agent to be taken up by macrophages and for the amount of the contrast agent in the lumen of the vessel to be imaged to be reduced to an amount which allows macrophages that may be present in the vessel wall to be visualized. Waiting this amount of time allows sufficient contrast between the lumen of the vessel and the cells of the vessel wall.

In one embodiment, multiple images may be taken after administration of the contrast agent. In one embodiment, an image is made while the contrast agent is in the lumen of the vessel to obtain a positive image of the vessel and a second image is made after the amount of the contrast agent in the lumen of the vessel to be imaged is reduced to an amount which allows macrophages that may be present in the vessel wall of the subject to be visualized to thereby obtain a positive image of phagocytes that may be present in the vessel wall.

In another embodiment, the improved contrast agent of the instant invention is used for blood pool imaging. In one embodiment, vascular scanning in vascular beds of interest (kidney, liver, heart, brain and elsewhere), may be performed.

Whole body vascular imaging, as well as imaging of specific sites, may be performed using routine imaging technology known to those of skill in the art, e.g., x-ray imaging, ultrasonagraphy, computed tomography (CT), computed tomography angiography (CTA), electron beam (EBT), magnetic resonance imaging (MRI), magnetic resonance angiography (MRA), and positron emission tomography.

In another embodiment, tumor vascularization can be monitored using the improved contrast agents of the invention. The invention provides methods for imaging the perfusion status, e.g., microperfusion status, of tumors, e.g., measurement of angiogenesis in tumors. The improved contrast agents of the invention may be used to identify cancerous tissue by, e.g., visualizing the diffusion of the contrast agent of the invention out of vessels surrounding a tumor.

In addition, the minimal diffusion of the contrast agents of the invention from intact intravascular space allows areas of vascular disease or disorder, or vascular damage, e.g., leakage (extravasation), at sites of, e.g., tissue damage or tumors, to be visualized due to the accumulation of the contrast agent in areas outside of the intravascular space.

In yet another embodiment, the improved contrast agents of the invention can be used to perform vascular imaging in the central nervous system, e.g., in the brain. Nanoparticles have been shown previously to be capable of passing through the blood-brain barrier (Kepan Gao, Xinguo Jiang. 2006. International journal of pharmaceutics vol. 310, no1-2, pp. 213-219), making them useful in imaging structures in the brain, e.g., blood flow, vascularization of brain tumors, anurysm, etc. The improved contrast agents of the invention may be used to diagnose the occurrence of stroke, determine the risk of stroke, or evaluate the effectiveness of treatment of stroke in a subject.

### I. DEFINITIONS

As used herein, the term inflammation refers to the complex response of tissues to, e.g., pathogens, irritants, or damaged cells. Inflammation involves the elaboration of factors and recruitment of immune cells, e.g., phagocytic cells, to the site of the inflammation. Inflammation may be acute or chronic. One of the cell types present at sites of inflammation is the macrophage.

As used herein, the term “macrophage” refers to the relatively long-lived phagocytic cell of mammalian tissues, derived from blood monocytes. Macrophages are involved in all stages of immune responses. Macrophages play an important role in the phagocytosis (digestion) of foreign bodies, such as bacteria, viruses, protozoa, tumor cells, cell debris and the like, as well as the release of chemical substances, such as cytokines, growth factors and the like, that stimulate other cells of the immune system. Macrophages are also involved in antigen presentation as well as tissue repair and wound healing. There are many types of macrophages, including aveolar and peritoneal macrophages, tissue macrophages (histiocytes), Kupffer cells of the liver and osteoclasts of the bone, all of which are within the scope of the invention. Macrophages may also further differentiate within chronic inflammatory lesions to epitheliod cells or may fuse to form foreign body giant cells (e.g., granulomas) or Langerhan giant cells.

The terms “vasculature,” “vessels,” and “circulatory system” are intended to include vessels through which blood circulates, including, but not limited to veins, arteries, arterioles, venules and capillaries. The blood vascular system is commonly divided into the macrovasculature (e.g., vessels having a diameter of >0.1 mm) and microvasculature (e.g., vessels having a diameter <0.1 mm). As used herein, the term “capillary” includes any one of the minute vessels that connect the arterioles (e.g., the smallest divisions of the arteries located between the muscular arteries and the capillaries) and venules (e.g., the minute vessels that collect blood from the capillary plexuses and join together to form veins), forming a network of nearly all parts of the body. Their walls act as semipermeable membranes for the interchange of various substances, including fluids, between the blood and tissue fluid. The average diameter of capillaries is usually between about 7 micrometers to 9 micrometers. Their length is usually about 0.25 mm to 1 mm, the later being characteristic of muscle tissue. In some instances, (e.g., the adrenal cortex, renal medulla), capillaries can be up to 50 mm long.

The term “vascular disease or disorder,” also commonly referred to as “cardiovascular disease, coronary heart disease [CHD] and coronary artery disease [CAD]” as used herein, refers to diseases or disorders affecting the vascular system, including the heart and blood vessels. A vascular disease or disorder includes diseases or disorders characterized by vascular dysfunction, including, for example, intravascular stenosis (narrowing) or occlusion (blockage) due to, for example, a build-up of plaque on the inner arterial walls, and diseases and disorders resulting therefrom. Also intended to be within the scope of the invention are thrombotic, or thromboembolic, events. The term “thrombotic or thromboembolic event” includes any disorder that involves a blockage or partial blockage of an artery or vein with a thrombosis. A thrombic or thrombolic event occurs when a clot forms and lodges within a blood vessel which may occur, for example, after a rupture of a vulnerable plaque. Examples of vascular diseases and disorders include, without limitation, atherosclerosis, CAD, MI, unstable angina, acute coronary syndrome, pulmonary embolism, transient ischemic attack, thrombosis (e.g., deep vein thrombosis, thrombotic occlusion and re-occlusion and peripheral vascular thrombosis), thromboembolism, e.g., venous thromboembolism, ischemia, stroke, peripheral vascular diseases, and transient ischemic attack.

As used herein, the term “vascular plaque,” also commonly referred to as “atheromas,” refers to the substance which builds up on the interior surface of the vessel wall, sometimes resulting in the narrowing of the vessel. Plaque is the common cause of CAD. Usually, plaque comprises fibrous connective tissue, lipids (e.g., fat) and cholesterol. Frequently deposits of calcium salts and other residual material may also be present. Plaque build-up results in the erosion of the vessel wall, diminished elasticity (e.g., stretchiness) of the vessel and eventual interference with blood flow. Blood clots may also form around the plaque deposits thus further interfering with blood flow. Plaque stability is classified into two categories based on the composition of the plaque. As used herein, the term “stable” or “inactive” plaques refers to those which are calcific or fibrous and do not present a risk of disruption or fragmentation. These types of plaques may cause anginal chest pain but rarely myocardial infarction in the subject. Alternatively, the term “vulnerable” or “active” plaque refers to those comprising a lipid pool covered with a thin fibrous cap. Within the fibrous cap is a dense infiltrate of smooth muscle cells, macrophages, and lymphocytes. Vulnerable plaques may not block arteries but may be ingrained in the arterial wall, so that they are undetectable. They may also be asymptomatic. Furthermore, vascular plaques are considered to be at a high risk of disruption. Disruption of the vulnerable plaque is a result of intrinsic and extrinsic factors, including biochemical, haemodynamic and biomechanical stresses resulting, for example, from blood flow, as well as inflammatory responses from such cells as, for example, macrophages.

As used herein, the language “subject” is intended to include human and non-human animals. In certain embodiments, the subject is a mammal, e.g., a primate, e.g., a human. Preferred human animals include a human patient suffering from, or prone to suffer from, a vascular disease, thrombotic disease, stroke, or cancer, e.g., tumors. The term “non-human animals” of the invention includes all vertebrates, e.g., mammals, e.g., rodents, e.g., mice, and non-mammals, such as non-human primates, sheep, dogs, cows, chickens, rabbits, amphibians, reptiles and the like. In one embodiment, a “subject” is at risk for rupture of vulnerable vascular plaque.

The phrase “pharmaceutically acceptable” is employed herein to refer to those nanoparticulate contrast agents of the present invention, compositions containing such contrast agents, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

As used herein the term “particle size” refers to the particle diameter (or for particles that are not spherical, the size of the largest dimension of the particle). When particle sizes are discussed herein they are presented in a volume or weight basis (e.g., not number-weighted) unless stated otherwise.

As used herein, the term “particle size distribution (particle size density)” refers to the range of particle sizes present in a composition. The particle size density can be quantified and a single value ascribed, e.g., the mean particle size, the mode particle size and the median particle size. The mean is the area under the particle size density curve. The modal value is that size where the greatest number of particles are located. The median is the value at which 50% of the particles are greater and 50% of the particles are smaller for the parameter being measured.

As used herein, the term “nanoparticulate” or “nanoparticle” refers to a composition comprising microscopic particles having a size measured in nanometers. In one embodiment, particles in the sub-micron range (less than 1 micron or 1000 nanometers) are referred to herein as “nanoparticles.”

### II. CONTRAST AGENTS OF THE INVENTION

The contrast agents of the present invention include substances that can be introduced, e.g., injected, into a structure, e.g., an organ, tissue, blood vessel, blood pool, or plaque, and, because of the difference in the absorption of detection medium, e.g., x-rays, radiowaves, soundwaves or the like, between the contrast agent and the structure, allow for detection, visualization, or enhanced visualization, e.g., radiographic or sonographic visualization, of the structure, e.g., the organ, tissue, blood vessel, blood pool, or plaque.

In one embodiment, a contrast agent of the invention is not superparamagnetic iron containing particles. Preferably, the contrast agents of the invention are crystalline nanparticulate agents.

Nanoparticulates can be made from a broad number of materials including acrylates, methacrylates, methylmethacrylates, cyanoacrylates, acrylamides, polyacetates, polyglycolates, polyanhydrates, polyorthoesters, gelatin, polysaccharides, albumin, polystyrenes, polyvinyls, polyacroleines, polyglutataldehydes, and derivatives, copolymers, and derivatives thereof. In one embodiment, gold may be used to make a nanoparticulate contrast agent of the invention. Monomer materials particularly suitable to fabricate biodegradable nanoparticles by emulsion polymerization in a continuous aqueous phase include methylmethacrylates, polyalkycyanoacrylates, hydroxyethylmethacrylates, methacrylate acid, ethylene glycol dimethacrylate, acrylamide, N,N′-bismethyleneacrylamide and 2-dimethylaminoethyl methacrylate. Other nanoparticulates are made by different techniques from N,N-L-lysinediylterephthalate, alkycyanoacrylate, polylactic acid, polylactic acid-polyglycolic acid-copolymer, and desolvated macromolecules or carbohydrates. Further, non-biodegradable materials can be used such as polystyrene, poly (vinylpyridine), polyacroleine and polyglutaraldehyde. Other exemplary compounds suitable for use in the methods of the invention include those compositions described in, for example, U.S. Pat. Nos. 5,322,679, 5,466,440, 5,518,187, 5,580,579, 5,718,388, 5,525,328, 5,260,478, 5,537,750, 5,488,133, and 5,466,433 the contents of which are hereby incorporated by reference in their entirety.

A summary of exemplary materials and fabrication methods for making nanoparticulates has previously been published. See Kreuter, J. (1991) “Nanoparticles-preparation and applications.” In: M. Donbrow (Ed.) “Microcapsules and nanoparticles in medicine and pharmacy.” CRC Press, Boca Ranton, Fla., pp. 125-148.

In one embodiment, the contrast agent used in the methods of the invention is an ester of diatrizoic acid. In another embodiment, the contrast agent used in the methods of the invention is an iodinated aroyloxy ester. In still another embodiment, the contrast agent used in the methods of the invention is N1177 (also referred to as WIN 67722 and PH-50). N1177 is an iodinated aroyloxy ester with the empirical formula C₁₉H₂₃I₃N₂O₆, and the chemical name 6-ethoxy-6-oxohexy-3,5-bis(acetylamino)-2,4,6-triiodobenzoate. N1177 is in a crystalline form and is milled in an aqueous millieu to generate nanoparticles and is non-soluble, e.g., non-water soluble.

In another embodiment, the contrast agents of the invention are non-water soluble. In still another embodiment, the contrast agents of the invention comprise, or are labeleable with, a heavy element, e.g., iodine or barium, which may or may not be radioactively labeled. For example, the concentration of the heavy element, e.g., iodine, may be in a 2:1 ratio of labelable compound to iodine. In still another embodiment, the contrast agents of the invention have a half-life in the vasculature of a subject of at least about 30 minutes. In yet another embodiment, the contrast agent has a neutral pH.

The nanoparticle contrast agents of the invention are present in compositions having a defined particle size distribution. For example, the compositions of the invention have a specified mean particle size and/or range of particle sizes.

One of skill in the art will appreciate that particle sizes and particle size distribution measurements may vary depending upon the technique used to determine the size (e.g., PCS vs XDC); the use of different techniques or different methodology on the same composition may yield different results. For example, the table below shows that, for a given formulation, the measured particle size varies depending on the specific parameter reported (i.e., mean size, voume weighted, number weighted, etc.).

Size of large and small formulations in nanometers (nm)

| | | PCS | PCS | flow field |
|---|---|---|---|---|
| | PCS | Dv ± sd | Dn ± sd | fractionation - |
| Formulation | z-ave ± sigma | Volume weighted | Number weighted | mean |
| Compound A | 269.3 ± 0.223 | 296 ± 49, 83% | 269 ± 75, 100% | 229.6 |
| | | 524 ± 98, 16% | | (Diameter between |
| | | | | 100 nm to 600 nm) |
| Compound B | 128.5 ± 0.379 | 30.6 ± 8, 50% | 27 ± 8, 99% | 140 |
| | | 106 ± 37, 14% | | (Diameter between |
| | | | | 80 nm to 350 nm) |

In some embodiments the particle size (e.g., the mean particle size) is measured using the PCS technique. In other embodiments, XDC is used to measure the particle size. In some specific embodiments the number-weighted (D_{N}) measurement is used to measure the particle size, while, in other embodiments the volume-weighted (D_{V}) measurement is used. When particle sizes are discussed herein they are presented in a volume or weight basis (e.g., not number-weighted) unless stated otherwise.

Thus, although the absolute size may differ somewhat depending upon the methodology used, the relative sizes of compound A and compound B are consistent and compositions of the invention having smaller particle sizes are have improved properties when compared to the compositions having larger particle sizes.

In a preferred embodiment, a contrast agent of the invention has a mean particle size of less than 150 nanometers (nm). In a preferred embodiment, a contrast agent of the invention has a mean particle size of less than 100 nm. In one embodiment, the mean particle size of a nanoparticulate contrast agent of the invention is between about 110 nm and 140 nm. In another embodiment, the mean particle size of a nanoparticulate contrast agent of the invention is about 130 nm. In another preferred embodiment, the contrast agent has a mean particle size between about 75 nm and 150 nm. In another embodiment, a contrast agent of the invention is a composition comprising a nanoparticulate contrast agent having a mean particle size of between about 20 nm and about 150 nm. In other embodiments, a contrast agent of the invention has a mean particle size of between about 20.0 nm and about 30 nm, between about 30 nm and about 40 nm, between about 40 nm and about 50 nm, between about 50 nm and about 60 nm, between about 60 nm and about 70 nm, between about 70 nm and about 80 nm, between about 80 nm and about 90 nm, between about 90 nm and about 100 nm, between about 100 nm and about 110 nm, between about 110 nm and about 120 nm, between about 120 nm and about 130 nm, between about 130 nm and about 140 nm, between about 140 nm and about 150 nm, between about 150 nm and about 160 nm, between about 160 nm and about 170 nm, between about 170 nm and about 180 nm, between about 180 nm and about 190 nm, between about 190 nm and about 200 nm. Also, in some embodiments, the a contrast agent of the invention has a mean particle size of between 40 nm and 500 nm. In other embodiments a contrast agent of the invention has a mean particle size of less than 500 nm, less than 400 nm, less than 300 nm, less than 250 nm, less than 200 nm, less than 170 nm, less than 150 nm, less than 130 nm, less than 110 nm, less than 100 nm, less than 95 nm, less than 90 nm, less than 80 nm, less than 70 nm, less than 60 nm, less than 50 nm, or less than 40 nm.

In one embodiment a nanoparticulate contrast agent of the invention is present in a composition comprising a particle size distribution in which 50% of the nanoparticles are not more than 100 nanometers in diameter and 90% of the nanoparticles are not more than 200 nanometers in diameter.

In another preferred embodiment, a nanoparticulate contrast agent of the invention is present in a composition comprising a nanoparticulate contrast agent having a particle size distribution wherein no more than 50% of nanoparticles are less than about 70 nm. In one embodiment, 100% of the nanoparticles are less than about 400 nm. In another embodiment, 100% of the nanoparticles are less than about 350 nm. In another embodiment, 100% of the nanoparticles are less than about 300 nm, 100% of the nanoparticles are less than about 250 nm. In another embodiment, 100% of the nanoparticles are less than about 200 nm. In another embodiment, 100% of the nanoparticles are less than about 150 nm. Alternatively, in some embodiments, 50% or 100% of the nanoparticles are greater than 50 nm, greater than 75 nm, greater than 90 nm, greater than 100 nm, greater than 125 nm, greater than 150 nm, or greater than 170 nm.

In some embodiments, a nanoparticulate contrast agent of the invention may be described according to both a mean particle size and a particle size range or distribution. Any of the mean particle sizes described herein which are compatible with particle size distributions described herein are envisioned. For example, in some embodiments the contrast agent has a mean particle size of about 150 nm or less and 100% of the nanoparticles are less than about 400 nm. In a related embodiment the contrast agent has a mean particle size of about 150 nm or less and 100% of the nanoparticles are less than about 300 nm. In further related embodiments the mean particles size is less than 100 nm and 100% of the nanoparticles are less than 300 nm, less than 400 nm, less than 500 nm, or less than 600 nm. In further related embodiments the mean particles size is less than 150 nm and 100% of the nanoparticles are less than 400 nm, less than 500 nm, or less than 600 nm. In further related embodiments the mean particles size is between 100 nm and 150 nm and 100% of the nanoparticles are less than 300 nm. In other embodiments the mean particles size is between 100 nm and 150 nm and 100% of the nanoparticles are less than 400 nm. In other embodiments the mean particles size is between 100 nm and 150 nm and 100% of the nanoparticles are less than 500 nm or less than 600 nm. In other embodiments the mean particles size is between 100 nm and 150 nm and a particle size distribution is between about 80 and about 350 nanometers in diameter as measured by asymmetrical flow field fractionation or of between about 20 and about 120 as measured by PCS.

As used herein, the term “about” with respect to particle size refers to a difference of +/−7 nm from the stated value.

Since the particle compositions of the invention contain a distribution of particle sizes, a statement about mean particle size (e.g., a mean of about 140 nm or a mean between 100 and 140 nm) may be accompanied by a standard deviation which expresses the breadth of the distribution. The standard deviation may be expressed as the range of sizes encompassing 80%, 90%, 95%, or 99% of the particles. In some embodiments, the standard deviation is 10 nm. In other embodiments, the standard deviation is 5 nm, 15 nm, 20 nm, 25 nm, 30 nm, 35 nm, or 40 nm, 45 nm, or 50 nm.

### III. METHODS OF MAKING CONTRAST AGENTS

Exemplary compounds suitable for use in the methods of the instant invention may be synthesized by methods known in the art.

Methods of making particles in the nanoparticulate size range are well known in the art and the size and size range of such particles in pharmaceutical compositions can be closely controlled. For example, the nanoparticulate contrast agents used in the methods of the invention may be produced by processes known in the art for the production of the desired particle size, or by methods described in, for example, U.S. Pat. Nos. 5,718,388, 5,518,187, 5,543,133 and 5,862,999 or US patent applications 20040169194 or 20040173696. Methods known in the art include, for example, high energy media milling, low energy ball milling, acoustic cavitation, hydrodynamic cavitation, as well as other means which reduce particle size via shear or impact.

In one embodiment, a contrast agent of the invention milled to the desired size. In a preferred embodiment the contrast agent to be prepared is N1177, which may be synthesized from a commercially available starting material, sodium diatrizoate (sodium 3,5-(acetylamino)-2,4,6-triiodobenzoate).

An exemplary synthesis of N1177 may begin with preparing a stirred slurry of equimolar sodium diatrizoate and ethyl 6-bromohexanoate in N,N-dimethylformamide, and heating the slurry to about 95° C. for, e.g., at least four hours. The precipitated solids may be collected by filtration and washed and dried. The expected yield of the crude synthesis is expected to be about 90%.

Crude N1177 may then be purified by stirring N1177 in a slurry of dimethyl sulfoxide which is heated in a nitrogen atmosphere. The resulting solution may be passed through a cartridge filter into distilled water. The precipitated solids may be filtered and washed with water and vacume dried at about 85° C. The dry material may be suspended in ethanol with agitation and heated to near reflux. The internal temperature may then be lowered to below 40° C. The suspension may then be filtered and washed with ethanol, yielding pure N1177 which may then be dried.

Once the crystallized N1177 has been synthesized, it may be milled to the desired mean particle size and distribution.

A nanoparticulate contrast agent of the invention may be formulated to improve its physical or pharmaceutical properties. In one embodiment, the N1177 is prepared as a suspension of crystalline iodinated particles dispersed with a biocompatible surfactant. The surfactant prevents particle aggregation and stabilized particle size. One preferred surfactant is poloxamer 338 which is added to N1177. A nanoparticulate suspension is obtained by milling the iodinated aroyloxy ester and poloxamer 338 in the presence of milling media. In a preferred procedure, a 200 ml dispersion of 150 mg/ml and 30 mg/ml is charged into a 1000 ml bottle containing 2 kg of Yttrium Stabilized Zirconia (YTZ) 0.5 mm milling media. In one embodiment, the sample is ball milled using a roller mill at 90 RPM for 1 to 7 days.

### IV. METHODS OF ASSESSING PARTICLE SIZE

The particle size of the agents of the invention may be measured using standard methods.

In one embodiment, XDC (X-ray disc centrifuge sedimentometry) is used to measure particle size distribution. This is one of the most precise methods available for inorganic materials. The technique is based on the fact that particles of a different size (and of the same density) will take different times to travel between two points under the same force (e.g., gravity, centrifugal force): a larger particle will travel faster than a small particle. The derivation of the Stokes' equation for centrifugal sedimentation has been described many times in the literature (Thomas, J. C., and Fairhurst, D. (1991) Surface Phenomena and Fine Particles in Water-based Coatings and Paints. Sharma, M. K., and Micale, F. J. (eds), Plenum Press, New York). The relationship between size and the time taken is a square function. Thus, if a particle of (for example) one micron, takes one second to traverse a given distance, a 0.1 micron particle will take 100 seconds. With a distribution a particle sizes, the particles will effectively fractionate as they move under the proscribed (centrifugal) force. Because time can be measured extremely precisely, the sizes detected can be measured with great precision. The method used to determine the “amount” of material at any given size is X-ray absorbtion. For many inorganic materials, X-rays are absorbed in proportion to mass. The particle size density is therefore “mass-weighted.” Since, for a given particle density, mass is proportional to volume, a “volume-weighted” value (Dᵥ) can be calculated for particle size density. Making the assumption that the particles are spherical, Dv can be transformed into either a surface-weighted value (Dₛ) or a number-weighted value (Dₙ). The latter number can be compared with the size determined from electron microscopy. For particle sizes in the colloidal domain (less than 1 micron), the assumption of sphericity is quite reasonable and becomes more so as the particle size gets smaller into the deep sub-micron (<100 nm) and the true nanometer (ca 10 nm) range. The size range that can be accommodated in XDC measurements is dictated only by the width of the particle size density, e.g., how long the measurement will take. The XDC method can be quite time consuming for a very broad particle size density.

In another embodiment, laser light scattering can be used to measure particle size.

This is the most widely used technique for particle size density determination and there are two main variations: photon correlation spectroscopy (PCS), and Fraunhofer Diffraction (FD). The choice is dictated by the particle size range under investigation. PCS works for sizes from a few nanometers up to about 1 micron (1000 nm), and FD works from about 1 micron up to millimeters. Both methods are called “ensemble averaging methods.” This means that the two relevant pieces of information needed to describe the particle size density (the actual size and the amount of material at that size) need to be deconvoluted from a single measurement of the amount of light scattered. This involves application of extremely complex theory and equally complex deconvolution algorithms (Kerker, M. (1969) The Scattering of Light and other Electromagnetic Radiation. Academic Press, New York). Thus, both measurement variations are intrinsically low resolution: typically the best that can be achieved is to differentiate between two class sizes (Weiner, B. B. (1984) Modern Methods of Particle Sizing. Barth, H. (ed). Wiley-Interscience). A technical issue arises when the particle size density extends across the one micron point, because then the two different algorithms/theories need to be applied and these cannot be combined together. Attempts to do so result in artifacts in the particle size density such that commercial instruments smooth the data resulting in even less resolution. Being based on light scattering, the fundamental value obtained is an “intensity-weighted” number (Dᵢ). Transforming the Dᵢ value into a Dᵥ value presents several challenges. The first is that, in addition to the assumption of sphereicity, both PCS and FD weight the intensity differently, and the second is that the transform necessitates application of scattering and efficiency corrections (Weiner, B. B., Fairhurst, D., and Tscharnuter, W. W. (1991) Particle Size Distribution II—Assessment and Characterization. Provder, T. (ed). ACS Symposium Series No. 472. American Chemical Society). Although such corrections can be calculated from Mie theory (Kerker, M. (1969) The Scattering of Light and other Electromagnetic Radiation. Academic Press, New York), they are particle size dependent and require knowledge of the optical properties (complex refractive index) of both the particles and the surrounding medium. The result is that the Dᵥ value (and if further transforms are made to Dₛ and Dₙ) can be widely inaccurate. However, for quality control purposes the technique is extremely fast and, for measurements on the same material, extremely reproducible. Laser light scattering may also be referred to as Dynamic Light Scattering (DLS) and generally refers to the PCS technique. Asymmetrical flow field-flow-fractionation (AF4) is a newer method based on light scattering which may be employed. The benefit A4F is that the method is able to separate nanoparticle mixtures prior to DLS analysis.

In one embodiment, a nanoparticulate contrast agent of the invention is present in a composition in which at least about 50% of the nanoparticles have a Dv of between about 15 nm and about 150 nm. In another embodiment, a nanoparticulate contrast agent of the invention is present in a composition in which at least about 50% of the nanoparticles have a Dv of between about 20 nm and about 130 nm. In another embodiment, a nanoparticulate contrast agent is present in a composition in which at least about 50% of the nanoparticles have a Dv of between about 25 nm and about 110 nm. In another embodiment, a nanoparticulate contrast agent is present in a composition in which at least about 50% of the nanoparticles have a Dv of between about 30 nm and about 100 nm. In another embodiment, a nanoparticulate contrast agent of the invention is present in a composition in which at least about 50% of the nanoparticles have a Dn of less than about 200 nm.

In one embodiment, a nanoparticulate contrast agent of the invention is present in a composition in which at least about 90% of the nanoparticles have a Dn of less than about 200 nm. In one embodiment, a nanoparticulate contrast agent of the invention is present in a composition in which at least about 90% of the nanoparticles have a Dn of less than about 100 nm. In one embodiment, a nanoparticulate contrast agent of the invention is present in a composition in which at least about 90% of the nanoparticles have a Dn of less than about 50 nm. In one embodiment, a nanoparticulate contrast agent of the invention is present in a composition in which at least about 90% of the nanoparticles have a Dn of about 30 nm.

In one embodiment, a nanoparticulate contrast agent of the invention is present in a composition in which at least about 90% of the nanoparticles have a Di of less than about 200 nm.

In another example, monitoring of particle size distributions can be performed using the methods described in (McFadyen, P., and Fairhurst, D. (1993) Clay Minerals 28:531-537 or Pecora, R. (1985) Dynamic Light Scattering—Applications of Photon Correlation Spectroscopy, Plenum Press. New York).

### V. CONJUGATION TO A PHARMACEUTICALLY ACTIVE AGENT

In one embodiment, a nanoparticluate contrast agent of the invention is conjugated to a pharmaceutically active agent.

As used herein, the term “pharmaceutically active agent” refers to any chemical substance, e.g., any drug or compound that is used in the treatment, cure, prevention, or diagnosis of a disease or disorder of a subject associated with disorder, e.g., an anti-inflammatory agent, another agent used in the treatment of coronary artery disease, or an anti-cancer agent. Examples of the form of pharmaceutically active agents which are included in the present invention include, without limitation, small molecules, peptides, ribozymes, antisense oligonucleotides, short interfering RNA (siRNA), radiopharmaceutical agents, naked nucleic acid or a nucleic acid molecule incorporated into a viral vector. By naked nucleic acid is meant an uncoated single or double stranded DNA or RNA molecule.

Exemplary anti-inflammatory agents include, e.g., phenylbutazone, indomethacin, naproxen, ibuprofen, flurbiprofen, diclofenac, dexamethasone, prednisone and prednisolone, histamine, bradykinin, kallidin and their respective agonists and antagonists, immune modulatory agents, anti-infective agents, lipid-lowering agents, cytokine modulating agents, anti-thrombogenic drugs, such as heparin or a heparin derivative, anti-proliferative drugs such as enoxaprin, angiopeptin, or antibodies, e.g., polyclonal antibodies or monoclonal antibodies, hirudin or acetylsalicylic acid (e.g., aspirin). Preferably, a pharmaceutically active agent is not simply used as a tareting means.

Exemplary anti-cancer agents include alkylating agents (e.g., Cisplatin, carboplatin, oxaliplatin, mechlorethamine, cyclophosphamide, chlorambucil), antimetabolites (e.g., azathioprine, mercaptopurine), plant alkaloids and terpenoids, vinca alkaloids (e.g., Vincristine, Vinblastine, Vinorelbine, Vindesine), Podophyllotoxin, etoposide, teniposide, paclitaxel (taxol), docetaxel, topoisomerase inhibitors (e.g., camptothecins, irinotecan, topotecan, amsacrine, etoposide, etoposide phosphate, teniposide), taxanes, dactinomycin, anthracyclines, doxorubicin, daunorubicin, epirubicin, bleomycin, plicamycin, mitomycin, anti-tumor antibodies, anti-angiogenesis drugs, and others known in the art.

In another embodiment, the pharmaceutically active agent is non-water soluble. In still another embodiment, the drug is a prodrug which is metabolically converted into an active agent once administered to the subject or once taken up by a mononuclear phagocyte, e.g., a macrophage. In a further embodiment, the pharmaceutically active agent is a sustained-release agent.

For example, in one embodiment, the nanoparticulate may be coated by one or more pharmaceutically active agents. The resulting pharmaceutical composition may be a sustained release formulation, e.g., it may provide for delivery of a pharmaceutically active agent over an extended period. Depending on the desired drug release properties, a nanoparticulate may be coated with a single layer of coating, or alternating coatings may be provided, or the pharmaceutically active agent may actually be interdispersed within a coating material (see, e.g., U.S. Pat. No. 6,406,745 and Modern Pharmaceutics, Second Edition, edited by Gilbert S. Banker and Christopher T Rhodes, the entire contents of which is hereby incorporated by reference). Materials used for the coating include most solids currently used in the pharmaceutical and food industries, namely any material that can be effectively ablated by the energy source. These materials include, but are not limited to, biodegradable and biocompatible polymers, polysaccharides, and proteins. Suitable biodegradable polymers include polylactides, polyglycolides, polycaprolactones, polydioxanones, polycarbonates, polyhydroxybutyrates, polyalkylene oxalates, polyanhydrides, polyamides, polyesteramides, polyurethanes, polyacetates, polyketals, polyorthocarbonates, polyphosphazenes, polyhydroxyvalerates, polyalkylene succinates, poly(malic acid), poly (amino acids), polyvinylpyrrolidone, polyethylene glycol, polyhydroxycellulose, polyorthoesters, and combinations thereof, as well as other polylactic acid polymers and copolymers, polyorthoesters, and polycaprolactones, etc. Suitable biocompatible polymers include polyethyleneglycols, polyvinylpyrrolidone, and polyvinylalcohols, etc. Suitable polysaccharides include dextrans, cellulose, xantham, chitins and chitosans, etc. Suitable proteins include polylysines and other polyamines, collagen, albumin, etc. A number of materials particularly useful as coating materials are disclosed in U.S. Pat. No. 5,702,716.

In another embodiment, the nanoparticulate is a hollow sphere, semi-sphere, or liposome in which one or more pharmaceutically active agents are encapsulated for delivery, for example, for sustained release delivery.

In a further embodiment, the nanoparticulate may be conjugated, e.g., covalently or non-covalently conjugated, to one or more pharmaceutically active agents as described in, for example, U.S. Pat. No. 6,482,439, or by methods known in the art. For example, it may be desirable to directly couple a pharmaceutically active agent and a nanoparticulate or to couple a pharmaceutically active agent and a nanoparticulate via a linker group or bridging agent. In one embodiment, the interaction between the nanoparticule and the pharmaceutically active agent is ionic. More than one pharmaceutically active agent may be coupled to a polyvalent nanoparticulate.

The pharmaceutically active agent can also either be adsorbed (or absorbed) to a pre-made nanoparticulate or it can be incorporated into the nanoparticulate during the manufacturing process. Methods of absorption, adsorption, and incorporation are common knowledge to those skilled in the art.

One or more oligonucleotides may also be associated with the nanoparticulates of the invention. For example, an oligonucleotide may have a functional group associated therewith which can bind to the nanoparticles. The nanoparticulates may be, for example, positively charged.

The pharmaceutically active agent itself, e.g., a non-water soluble agent, may be formulated as a nanoparticulate for administration, e.g., by methods known in the art and described herein for preparation of nanoparticulates. In a further embodiment, the nanoparticulate drug delivery vehicle may be enzymatically degradable. Upon administration of an enzymatically degradable nanoparticulate to a subject, the nanoparticulate composition is degraded, leaving the pharmaceutically active agent.

### VI. METHODS OF USE

As set forth in more detail below, the compositions of the instant invention can be used to image the lumen of a blood vessel or to image sites of inflammation by facilitating the detection of phagocytic cells present in the wall of a blood vessel or in the extravascular space.

Various experiments demonstrate that particle size has a significant effect upon the clearance of contrast agents from the blood. For example, in some embodiments, smaller particle sizes result in longer blood enhancement (e.g., longer half life and slower clearance rate) as compared to larger particle sizes. In some embodiments, the contrast agents of the invention have a beta half life of up to 3 hours. In other embodiments the contrast agent has a half live between 2.5 hrs and 3 hours, between 2 hrs and 2.5 hrs, between 1.5 hrs and 2 hrs, between 1 hr and 1.5 hrs, between 0.5 hrs and 1 hr, or between 0 hrs and 0.5 hrs. In specific embodiments, the contrast agents of the invention have a half life of around 1 hour, e.g., 1.12 hrs, 0.97 hrs, or 1 hr±up to 0.4 hrs (e.g., 1.1 hrs, 0.9 hrs, 1.05 hrs, 0.97 hrs, and all such sums or differences of 1 hr with any time interval up to 0.4 hrs). In other embodiments the contrast agents of the invention have a half life between 0.3 and 0.5 hrs, between 0.4 hrs and 0.5 hrs, between 0.5 and 0.6 hrs, between 1.4 and 1.5 hrs, or between 1.5 and 1.6 hrs. Depending on the situation shorter or longer half lives may be desired. In some embodiments the contrast agents of the invention have a half life of less than 0.5 hrs, less, than 1 hr, less than 1.5 hrs. In other embodiments, the contrast agents of the invention have a half life of more than 0.2 hrs, more than 0.5 hrs, more than 0.7 hrs, more than 1 hr, more than 1.5 hrs, more than 2 hrs, more than 2.5 hrs, or more than 3 hrs.

In one embodiment, after administration of a composition of the invention, an image is made after a period of time equal to or greater than the half life of the contrast agent in the circulation, such that a positive image of phagocytic cells can be made (e.g., such that the image of the phagocytic cells can be seen against any contrast remaining in the lumen of the vessel).

In another embodiment of the invention, an image is made after a period of time less than or equal to the half life of the contrast agent in the circulation, such that a positive image of the lumen of the vessel can be made (e.g., such that the image of the contrast medium in the lumen of the vessel can be seen against the background).

In one embodiment, the agent is administered by being injected intravenously or intra-arterially, whereupon imaging of the vasculature can be achieved by using standard imaging techniques. The invention provides for visualization, e.g., detection or imaging, of the contrast agent using any imaging techniques which are well-known in the art. These techniques may include, but are not limited to, x-ray imaging, ultrasonagraphy, computed tomography (CT), computed tomography angiography (CTA), electron beam (EBT), magnetic resonance imaging (MRI), magnetic resonance angiography (MRA), and positron emission tomography. Preferably, the detection is by CT.

A. Macrophages and Imaging Vascular Inflammation

Recent evidence suggests that inflammation in the vasculature, such as the coronary arteries, may be intimately involved in the development of many diseases, such as cancer and atherosclerosis and its associated acute coronary syndromes. As a part of the inflammatory response, phagocytic cells (e.g., macrophages) migrate to and accumulate at the site of inflammation. Accordingly, one aspect of the invention provides a method of obtaining or evaluating and image of accumulated macrophages associated with inflammation in a blood vessel (e.g., in an artery such as a coronary or pulmonary artery or a vein) or in tissue by administering, e.g., intravenously, to a subject, e.g., a mammal, such as a human, an effective amount of a contrast agent so as to detect the agent and form an image of the accumulated macrophages in the vessel after waiting for a period of time sufficient for uptake of the contrast agent by the macrophages and a time sufficient for enough of the contrast agent to be cleared from the lumen of the vessel such that a positive image of the macrophages can be observed.

Furthermore, the invention includes methods for detecting ischemic, inflamed, injured, or infected tissues, or vessels, vascular wall damage, and the like, using the contrast agents of the invention based on the imaging and detection of phagocytes, e.g., activated macrophages, at the site of, e.g., ischemia, inflammation, injury, or infection based on detection of accumulated macrophages. In another embodiment, phagocyte accumulation in extravascular space may also be detected. Where the contrast agent of the invention is present in the extravascular space due to, e.g., leakage, abscess, gaps, or lesions of the vascular wall, accumulation of phagocytic cells may be detected in areas of ischemia, inflammation, injury, or infection based on detection of accumulated phagocytes. Accordingly, inflammation, or inflammatory diseases or disorders, such as, but not limited to, infection, rheumatoid arthritis, chronic pulmonary inflammatory disease, psoriasis, rheumatoid spondylitis, osteoarthritis and gouty arthritis, deep vein thrombosis, allergy, multiple sclerosis, autoimmune diabetes, autoimmune diseases or disorders, nephrotic syndrome, cancer, atherosclerosis may be detected or diagnosed.

Moreover, healing or progress of treatment of tissues or vessels, or areas in the extravascular space, may also be visualized by the methods of the invention by imaging the accumulation of phagocytes at the injured site prior to treatment and post-treatment and determining whether the extent of inflammation has decreased with treatment.

The subject compositions are of particular value in detecting vulnerable atherosclerotic plaque. Whereas stable plaque is calcified and relatively free of inflammation, vulnerable plaque contains many phagocytic cells and, therefore, is readily detected. It is possible that the contrast agents of the invention enter inflamed tissue around vulnerable plaques, at least in part, through gaps in the endothelial cell layer surrounding these types of plaques.

Accordingly, yet another aspect of the invention pertains to a method of obtaining or evaluating (e.g., to determine whether plaque is present in the image) an image of plaque, e.g., vulnerable plaque, accumulation in a vessel, tissue, or organ of a subject by administering, e.g., intravenously, an effective amount of a contrast agent of the present invention to the subject and detecting phagocytes indicative of inflammation, e.g., associated with plaque accumulation, in the vessels.

The present invention also pertains to an imaging method for predicting risk of vascular disease by obtaining or evaluating an image of accumulated phagocytes within a blood vessel of a subject by administering an effective amount of the contrast agent of the present invention, detecting the agent within the subject and, based on the image obtained, predicting the risk of vascular disease in the subject. As used herein, the terms “predicting risk” and “prognosticating” refers to the assessment for a subject of the probability of developing a condition, e.g., vascular disease such as, but not limited to, atherosclerosis, coronary artery disease (CAD), myocardial infarction (MI), ischemia, stroke, peripheral vascular disease, and venous thromboembolism, rupture of vulnerable vascular plaque, or a stage associated with or otherwise indicated by assessment of an image obtained from the subject administered with a contrast agent of the present invention. Recent experimental and clinical studies based on the biochemcial markers of inflammation and vascular perturbation in plasma as well as in atherosclerotic tissue suggest a potential role for using biochemical markers and/or other indicators of inflammation as indicators of vascular disease (see, e.g., Van Lente, F. supra; and Schmidt, M. I. et al., supra). Accordingly, using the imaging data obtained from the methods of the present invention to image macrophages, together with other criteria such as age, obesity, cholesterol level, HDL and LDL levels, smoking, and the like which are well known to those skilled in the art, one skilled in the art will be able to predict the likelihood that the subject will develop a vascular disease or disorder or is at risk for developing a vascular disease or disorder. For example, a subject showing large macrophage accumulation together with high cholesterol and LDL levels will be at a greater risk than a subject showing little or no macrophage accumulation and low LDL levels. Imaging phagocyte accumulation according to the methods of the present invention can also assist in predicting, diagnosing, or prognosticating other vascular diseases or related disorders. Such other diseases include atherosclerosis, CAD, MI, unstable angina, acute coronary syndrome, pulmonary embolism, transient ischemic attack, thrombosis (e.g., deep vein thrombosis, thrombotic occlusion and re-occlusion and peripheral vascular thrombosis), thromboembolism, e.g., venous thromboembolism, ischemia, stroke, peripheral vascular diseases, and transient ischemic attack.

In one embodiment, the contrast agents of the invention may be used to diagnose the occurrence of stroke or to determine the risk of stroke in a subject. The contrast agents of the invention may be used to pinpoint quickly the precise location of a stroke and determine the extent of damage, to assess the blood flow throughout the brain, to distinguish between an ischemic or hemorrhagic stroke, to determine the extent of damage, to determine the present of regarding collateral (alternative) blood vessels in the brain, or to diagnose blockage in the carotid arteries. Thrombotic stroke is due to the formation of a clot, which typically occurs at the site of an atherosclerotic plaque. Such thrombotic clots may cause embolic stroke (i.e., the blockage of an artery) if the clot breaks away and blocks a blood vessel. Accordingly, the contrast agents of the invention may further be used to determine the risk of thrombotic or embolic stroke in a subject.

In another embodiment, several imaging procedures may be performed following a single administration of the contrast agent of the invention, e.g., N1177. For example, assessment of the risk for or presence of vascular disease may be carried out by imaging anatomy and structure of the vessels, e.g., coronary angiography, imaging of tissue perfusion, or imaging of cavities, e.g., heart cavities, and imaging of vascular inflammation during one imaging session. Furthermore, the lack of diffusion of the contrast agents of the invention out of intact vascular space also allows for whole body vascular imaging as well as imaging of whole body plaque burden, using routine imaging technology known to those of skill in the art.

B. Angiography

The invention further pertains to angiography or blood pool imaging, in particular vascular blood pool imaging (e.g., cardiac blood pool, aorta blood pool, vena cava blood pool, liver blood pool, etc.). Angiography or arteriography is a medical imaging technique in which an image is taken to visualize the inside or lumen of blood vessles and organs of the body. Although the compositions of the invention are particularly useful in imaging phagocytic cells in vessel walls, they are also useful in imaging the lumen of veins or arteries or the chambers of the heart.

The contrast agents of the invention are well suited for imaging blood pools in several organs. In one embodiment, the contrast agents of the invention are used in cardiac blood pool imaging, e.g., gated cardiac blood pool imaging or in a multiple-gated acquisition (MUGA) scan, or in blood pool imaging of other organs, e.g., splenic blood pool imaging, hepatic (liver) blood pool imaging, lung blood pool imaging, brain blood pool imaging, a pancreatic blood pool, or any other organ or tissue. In gated cardiac blood pool imaging, images are acquired during repeated cardiac cycles, typically at specific times in the cycle (using, e.g., an electrocardiographic synchronizer or gating device). The acquired images may be averaged over defined time intervals. Cardiac blood pool images may be focused on a specific chamber of the heart, e.g., the left ventricle, the right ventrical, the left atrium, or the right atrium.

In one embodiment, the contrast agents of the present invention can be used for angiography to diagnose, e.g., blockage of an artery, e.g., a peripheral artery, a coronary artery, or kidney arteries. Angiography can identify the exact location of the blockage and can assess the severity of the blockage, based on the image generated. Occlusions may also be detected as well as the percent of blockage of the artery. Angiography may also detect the presence of an aneurysm and may be used prior to surgery to assess the location and severity of the aneurysm.

C. Perfusion

The invention can be used to image microperfusion in organ tissues to assess the perfusion status of organs on the level of the smallest blood vessels, e.g., capillaries. Tissues and organs, e.g., kidneys, liver, brain, and lung, can be monitored for adequate blood supply and blood perfusion (e.g., blood pool imaging). This ability can be used in assessing organ damage associated with angina pectoris or heart attacks, stroke, or vascular damage or injury, thereby replacing the currently utilized Technetium99 scans, or the imaging of brain perfusion to assess pathological events (stroke, tumors, and the like), to assess vessel leakages (aneurisms and diffuse bleedings after trauma or other pathological events), or to determine the microperfusion status of tumors including monitoring of treatment effects for all these applications (including the effectiveness of anti-angiogenic treatment, surgical intervention, and other treatments). Furthermore, vessels may be imaged in order to assess occlusion due to build-up of plaque and assess the necessity of surgical procedures, e.g., bypass surgery or other invasive or non-invasive treatment, e.g., lifestyle changes, including, for example, changes in diet, or medication. Imaging contrast in small blood vessels is indicative of an active perfusion of these tissue areas and allows important conclusions on the health and viability of the tissue that is being imaged.

D. Cancer

The formation of a tumor-associated vasculature (i.e., tumor angiogenesis) has emerged as a critical step in tumor initiation, promoting local tumor progression and metastatic spreading. Solid cancers often show typical signs of inflammation and are infiltrated by many leukocyte populations, i.e., neutrophils, eosinophils, basophils, monocytes/macrophages, dendritic cells, natural killer cells, and lymphocytes. Ruegg, C., 2006. Journal of Leukocyte Biology. 2006; 80:682-684. Indeed, a causal relationship between chronic inflammation and cancer formation has been proposed. Interestingly, inflammation functions at all three stages of tumor development: initiation, progression and metastasis. In one embodiment, the invention provides a method of detecting the likelihood that a patient will develop a tumor or detects an early stage tumor by detecting inflammation in a subject. In another embodiment, the invention provides a method of detecting an established tumor by detecting inflammation in a subject. In yet another embodiment, the invention provides a method of detecting tumor metasteses by detecting inflammation in a subject.

Furthermore, the invention provides methods for imaging the perfusion status, e.g., microperfusion status, of tumors, e.g., measurement of angiogenesis in tumors. The growth of tumors to a clinically relevant size is dependent upon an adequate blood supply. This is achieved by the process of tumor stroma generation where the formation of new capillaries is a central event. Progressive recruitment of blood vessels to the tumor site and reciprocal support of tumor expansion by the resulting neovasculature are thought to result in a self-perpetuating loop helping to drive the growth of solid tumors. The development of new vasculature also allows an ‘evacuation route’ for metastatically-competent tumor cells, enabling them to depart from the primary site and colonize initially unaffected organs. Imaging of vessels, including capillaries, within or in the area of a tumor-like mass or growth provides a method to assess or diagnose whether the mass is in fact a tumor as opposed to a non-cancerous growth, e.g., a cyst, and also provides a method to determine whether a tumor is benign or malignant and if malignant, determining the degree of malignancy based on the degree of angiogenesis of the mass. Accordingly, in one embodiment, the invention provides a method of detecting a tumor by measuring tumor angiogenesis using a composition of the invention. For example, in one embodiment, a composition of the invention is administered and the degree of angiogenesis is measured by making an image of the vasculature at a site in a subject.

Furthermore, it has been established that the microvessels of tumors are particularly “leaky,” with permeability being high compared to the microvessel of non-tumorous, healthy and intact tissues. Therefore, the contrast agents of the invention may be used to identify tumorous tissue based on visualization of the diffusion status, or “leakiness” of the contrast agent of the invention out of vessels surrounding a tumor.

E. Neurological Disorders

Amyloid plaques appear early during Alzheimer's disease (AD), and their development is intimately linked to activated astrocytes and microglia. It has been shown that microglia are attracted to new Alzhimers plaques within a day of their formation (Meyer-Luehmann et al. Nature. 2008 Feb. 7; 451(7179):720-4; Masliah E. Nature. 2008 Feb. 7; 451(7179):638-9); Fiala et al. Alzheimers Dis. 2007 July; 11(4):457-63; Britschgi M, Wyss-Coray T. Nat. Med. 2007 April; 13(4):408-9). Microglia are immune cells which act upon infectious agents, damaged cells, or plaques in the brain. Microglia are present in sites of inflammation in the brain, and induce inflammation by modes similar to other phagocytic cells. Contrast agents of the invention may be taken up by microglia in the nervous system, allowing detection of sites of inflammation in the brain and elsewhere (e.g., amyloid plaques). Thus, the contrast agents of the invention may be used to diagnose the presence of Alzheimers' plaques in a subject or determine the extent of plaque formation in a subject known to have Alzheimers' disease, or a related neurological disease.

F. Determining the Effectiveness of Treatment

Images made using a composition of the invention may also be used to monitor the course of therapy for inflammation, e.g., therapy for atherosclerosis or anti-vascular (anti-angiogenesis) therapy or other cancer therapies in a subject, wherein a decrease in, e.g., inflammation or angiogeneis in the subject indicates effectiveness of the tumor therapy. The method of assessing the effectiveness of therapy may making a single image of the subject, but more likely includes making two or more images of the subject over a period of time, e.g., during the course of therapy or before and after completion of therapy. Furthermore, the contrast agents of the invention may be used to assess successful surgical treatment by assessing the presence or absence of inflammation or angiogenesis post-surgery.

### VII. IMAGING TECHNOLOGY USED IN THE METHODS OF THE INVENTION

As used herein, the term “imaging” or “clinical imaging” refers to the use of any imaging technology to visualize a structure, e.g., a blood vessel, e.g., a capillary, blood pool, or plaque, either in vivo or ex vivo by measuring the differences in absorption of energy transmitted by or absorbed by the tissue. Imaging technology includes x-ray technology, scanning thermography such as ultrasonagraphy, computed tomography (CT), multi-detector CT (MDCT), magnetic resonance (MRI or NMR), and radionucleotides, e.g., ¹²³I or ¹²⁵I, for use in techniques such as positron emission tomography and the like.

CT imaging involves measuring the radiodensity of matter. Radiodensity is typically expressed in Hounsefield Units (HU). Hounsefield Units are a measure of the relative absorption of computed tomography X-rays by matter and is directly proportional to electron density. Water has been arbitrarily assigned a value of 0 HU, air a value of −1000 HU, and dense cortical bone a value of 1000 HU. Conventional CT scanners produce a narrow beam of x rays that passes through the subject and is picked up by a row of detectors on the opposite side. The tube and detectors are positioned on opposite sides of a ring that rotates around the patient, although the tube is unable to rotate continuously. After each rotation the scanner must stop and rotate in the opposite direction. Each rotation acquires an axial image of approximately 1 cm in thickness, at approximately 1 second per rotation. The table moves the patient a set distance through the scanner. Spiral (helical) CT scanners comprise a rotating tube, which allows a shorter scan time and more closely spaced scans. Angiography is possible with spiral scanning. Multislice CT scanners are considered “supercharged” spiral scanners. Where conventional and spiral scanners use a single row of detectors to pick up the x-ray beam, multislice scanners have up to eight active rows of detectors. Multislice scanners give faster coverage of a given volume of tissue. Various types of CT technology used in clinical practice is described in, for example, Garvey, C. and Hanlon, R. (2002) BMJ 324:1077.

In CTA, iodinated contrast agents are injected intravenously and images are obtained. Highly detailed images of the vasculature are generally obtained using CTA by reformatting the axial images to yield a composite picture of the vessels. During this reformatting, the picture of the vasculature is optimized based on the measured density in the vessels being visualized. To perform this imaging, various baseline image subtractions are performed.

CT imaging techniques which are employed are conventional and are described, for example, in Computed Body Tomography, Lee, J. K. T., Sagel, S. S., and Stanley, R. J., eds., 1983, Ravens Press, New York, N.Y., especially the first two chapters thereof entitled “Physical Principles and Instrumentation”, Ter-Pogossian, M. M., and “Techniques”, Aronberg, D. J., the disclosures of which are incorporated by reference herein in their entirety.

In one embodiment, the methods of the invention are carried out by the following procedure. A series of CT images is acquired with appropriate temporal resolution beginning just prior to contrast medium administration and continuing through the period of contrast agent administration (1-30 seconds, 1 minute, 5 minutes, 10 minutes, 15 minutes, 20 minutes, 30 minutes, 40 minutes, 50 minutes, 60 minutes, 90 minutes, 120 minutes, or more) and for a selected time period after the administration. In another embodiment, imaging is carried out after administration of the contrast agent. A wide range of image acquisition periods can be used in the method of the invention.

For example, in one embodiment, the selected time period is from about 10 seconds postcontrast to about 10 hours postcontrast, from about 30 seconds postcontrast to about 3 hours postcontrast, more preferably from about 50 seconds postcontrast to about 1 hour postcontrast, or more preferably still from about 1 minute postcontrast to about 10 minutes postcontrast. In another embodiment, the selected time period is from the time of completion of the contrast agent to about 30, 40, 50, 60 seconds postcontrast, to about 5, 10, 15, 20, 30, 40, 50, 60 minutes postcontrast, or to about 1, 2, 3, 4, 5, 6, 7, 8, 9, or more hours post contrast. Multiple images or series of images may be taken after a single administration of a contrast agent of the invention, e.g., N1177.

A typical series might include an image every five seconds before and during the contrast medium administration, slowing further to an image every ten seconds for the subsequent three minutes, and finally slowing to an image every 30 seconds until the 10 minute completion of the series. These serial images are used to generate the dynamic enhancement data from the tissue and from the blood as measured in a vessel to be used for kinetic modeling and, ultimately, to the calculation of blood volume and perfusion within the tissue of interest. After the completion of the dynamic acquisition localized to the region-of-interest, it may be elected to acquire additional CT images of the patient in other anatomic sites to extract additional diagnostic data or for delayed images in the same site. After CT scanning, the subject is removed from the scanner unit, and the intravenous catheter used for injection of the contrast agent can be removed. The data acquired from the CT imaging procedure is processed to provide the necessary information.

The contrast enhanced CT images can be used, for example, to define the location, caliber, and flow characteristics of vascular structures within the scanned anatomic regions as well as macrophage accumulation and plaque accumulation. Moreover, the images can be utilized to monitor the effect of potentially therapeutic drugs which are expected to alter perfusion status, e.g., microvascular perfusion status.

The methods described herein are useful with substantially any tissue type. In one embodiment, the tissue is a member selected from the group consisting of normal tissue, diseased tissue, and combinations thereof. In a further preferred embodiment, the tissue is at least partially a diseased tissue and the diseased tissue is a member selected from the group consisting of tissues which are neoplastic, ischemic, hyperplastic, dysplastic, inflamed, traumatized, infarcted, necrotic, infected, healing and combinations thereof.

### VIII. PHARMACEUTICAL COMPOSITIONS

Another aspect of the present invention provides pharmaceutically-acceptable compositions which comprise a nanoparticulate contrast agent formulated with one or more pharmaceutically-acceptable carrier(s), in an amount effective to allow imaging of blood pools, vascular tissue perfusion and the extravasation of blood out of vessels, to detect macrophages, or to detect plaques, e.g., vulnerable plaque, within the vessels of a subject.

In a particular embodiment, the nanoparticulate contrast agent is administered to the subject using a pharmaceutically-acceptable formulation, e.g., a pharmaceutically-acceptable formulation that suitable for administration in liquid form, including parenteral administration, for example, by intravenous injection, either as a bolus or by gradual infusion over time, intraperitoneally, intramuscularly, intracavity, subcutaneously, transdermally, dermally or directed directly into the vascular tissue of interest as, for example, a sterile solution or suspension.

In one embodiment, N1177 formulated for use as a contrast agent comprises 150 mg/ml N1177, 150 mg/ml polyethylene glycol 1450NF, 30 mg/ml poloxamer 338. In addition, 0.36 mg/ml tromethamine, sufficient to buffer to neutral pH, is also used. In one embodiment, the pH of N1177 may be about 7.4.

The polymeric excipients poloxamer 338 and polyethylene glycol 1450, serve as particle stabilizers and are also intended to retard the rate of plasma clearance of particles by the reticuloendothelial system (RES) after intravascular administration. Poloxamer 338 is purified by diafiltration as a part of the manufacturing process to reduce the level of low-molecular weight polymer. Other appropriate excipients or particle stabilizers may also be used. Exemplary formulations can be found, e.g., in U.S. patent application 20070141159.

A composition comprising a nanoparticulate contrast agent of the invention may comprise a pharmaceutically acceptable carrier. The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as poloxamer 338 and polyethylene glycol 1450; (10) oils, such as peanut oil, cottonseed oil, safflower oil, sesame oil, olive oil, corn oil, and soybean oil; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Methods of preparing these compositions may include the step of bringing into association a nanoparticulate contrast agent with the carrier and, optionally, one or more accessory ingredients. Usually, the formulations are prepared by uniformly and intimately bringing into association a contrast agent with liquid carriers.

Liquid dosage forms for oral administration of the contrast agent(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active nanoparticulate contrast agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more contrast agent(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Pharmaceutical compositions of this invention suitable for parenteral administration comprise one or more contrast agent(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, e.g., F68 or F108.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of the contrast agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of nanoparticulate contrast agent(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the nanoparticulate contrast agent(s) is administered as a pharmaceutical, to humans and animals, it can be given per se or as a pharmaceutical composition containing, for example, 0.1 to 99.5% (more preferably, 0.5 to 90%) of active ingredient in combination with a pharmaceutically-acceptable carrier.

The term “administration” or “administering” is intended to include routes of introducing the nanoparticulate contrast agent(s) to a subject to perform their intended function. Examples of routes of administration which can be used include, for example, injection (subcutaneous, intravenous, parenterally, intraperitoneally, intrathecal. The pharmaceutical preparations are, of course, given by forms suitable for each administration route. For example, these preparations are administered, for example, by injection. The injection can be bolus or can be continuous infusion. Depending on the route of administration, the nanoparticulate contrast agent can be coated with or disposed in a selected material to protect it from natural conditions which may detrimentally effect its ability to perform its intended function. The nanoparticulate contrast agent can be administered alone, or in conjunction with either another agent as described above or with a pharmaceutically-acceptable carrier, or both. The nanoparticulate contrast agent can be administered prior to the administration of the other agent, simultaneously with the agent, or after the administration of the agent. Furthermore, the nanoparticulate contrast agent can also be administered in a proform which is converted into its active metabolite, or more active metabolite in vivo.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, transdermal, subcutaneous, intrasternal injection, and infusion. A preferred method of administration is intravenous.

The phrases “systemic administration,” “administered systemically”, “peripheral administration” and “administered peripherally” as used herein mean the administration of a nanoparticulate contrast agent(s), drug or other material, such that it enters the patient's system and, thus, is subject to metabolism and other like processes, for example, subcutaneous administration.

Regardless of the route of administration selected, the nanoparticulate contrast agent(s), which may be used in a suitable hydrated form, and/or the pharmaceutical compositions of the present invention, are formulated into pharmaceutically-acceptable dosage forms by conventional methods known to those of skill in the art.

To use the nanoparticulate contrast agents of the present invention, the contrast agent is given in a dose which is diagnostically effective. A “diagnostically effective amount” or “effective amount” of a nanoparticulate contrast agent of the present invention is typically an amount such that when administered in a physiologically tolerable composition is sufficient to enable detection of vascular sites, macrophage accumulation, and/or plaque, e.g., vulnerable plaque, within the subject.

### IX. DOSING

Typical dosages can be administered based on body weight, and typically are in the range of about 0.1 mL/kg to about 8.0 mL/kg, about 0.2 mL/kg to about 7.0 mL/kg, about 0.3 mL/kg to about 6.0 mL/kg, about 4 mL/kg to about 5.5 mL/kg, about 0.5 mL/kg to about 4.0 mL/kg, about 0.6 mL/kg to about 3.5 mL/kg, about 0.7 mL/kg to about 3.0 mL/kg, about 0.8 mL/kg to about 2.5 mL/kg, about 0.9 mL/kg to about 2.0 mL/kg, or about 1.0 mL/kg to about 1.5 mL/kg, based on a stock solution of about 150 mg/mL consisting of about 15% weight/volume [w/v].

In a preferred embodiment the applied dosage is within the range of 125 mg/kg to 250 mg/kg. In another embodiment, the applied dosage is less than or equal to about 125 m/kg. The administration of the contrast agent of the invention may be over a period of time, e.g., by infusion, or by a single administration. In one embodiment, the administration rate of the contrast agent is about 0.6 mL/sec to about 3 mL/sec.

The dosage of the nanoparticulate contrast may also vary with the radioactivity of a radioisotope and will be taken into account in determining a suitable dose to be given of the contrast agent of the present invention. For example, the mean lethal dosages of both ¹²⁵I and ¹²³I have been calculated at about 79+/−9 cGy (in Chinese hamster ovary cells; see, e.g., Makrigiorgos, et al. Radiat. Res. 11:532-544). For diagnostic purposes, the dosage will be less than the mean lethal dose for the radioisotope.

For example, with respect to the half-life of common radioisotopes, the half-life of ¹²³I at a dose between 1 and 20 mCi is about 13 hours, while the half-life of ¹³¹I at a dose of less than 5 mC is about 8 days. It is expected that a useful dose of ¹²³I-labeled contrast agent would be between 1 and 20 mCi, while less than 5 mCi of the longer-lived ¹³¹I would be used (e.g., 0.5-5 mCi). Thus, for use according to the present invention, the preferred dose of agents including radioisotopes with longer half-lives will be less than the preferred dose of agents including radioisotopes with shorter half-lives.

Compositions comprising the nanoparticulate contrast agent are conventionally administered intravenously, as by injection of a unit dose, for example. The term “unit dose” when used in reference to the nanoparticulate contrast agent of the present invention refers to physically discrete units suitable as unitary dosage for the subject, each unit containing a predetermined quantity of active material calculated to produce the desired effect in association with the required diluent, e.g., carrier, or vehicle. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a desired effect. Generally, out of one hundred percent, this amount will range from about 1 percent to about ninety-nine percent of active ingredient, preferably from about 5 percent to about 70 percent, most preferably from about 10 percent to about 30 percent.

The nanoparticulate contrast agent is administered in a manner compatible with the dosage formulation, and in an effective amount. The quantity to be administered depends on the subject, capacity of the subject's system to utilize the active ingredient, the degree of contrast desired, and the structure to be imaged. Precise amounts of the contrast agent required to be administered depend on the judgement of the practitioner and are peculiar to each individual. However, suitable dosage ranges for systemic application are disclosed herein and depend on the route of administration. Suitable regimes for initial administration and subsequent administration, e.g., after initial imaging, are also contemplated and are typified by an initial administration followed by repeated doses at one or more hour intervals by a subsequent injection or other administration. Bolus administration, multiple dosages or continuous intravenous infusion sufficient to maintain concentrations in the blood in the ranges for specific in vivo imaging are also contemplated. Infusion of the contrast agent may be for less than one minute, two minutes, three minutes, four minutes, five minutes, or more.

### X. KITS

It is anticipated that the methods and the contrast agents of the invention can be incorporated into a commercial kit or system for imaging, detecting, and evaluating the perfusion and extravasation of blood out of vascular tissue, including but not limited to, vascular beds (e.g., arterial and venous beds), organ tissues (e.g., myocardial tissues and other organ tissues), and tumors, e.g., for the measurement of angiogenesis or perfusion status of tumors, or for the imaging, detecting, and evaluating phagocyte accumulation or plaque accumulation. Moreover, the method and contrast agents of the invention can be incorporated into a kit for determining the changes in tissue perfusion or microperfusion, angiogenesis, extravasation of blood, macrophage accumulation, or plaque accumulation, in response to treatment measures. The kit may contain a nanoparticulate contrast agent, and instructions for use and may further contain directions on the administration and use of the nanoparticulate contrast agent in conjunction with the appropriate imaging technology and dosage requirement for the intended use.

Other features, advantages and embodiments of the invention will be apparent from the following examples which are meant to illustrative, and therefore, not limiting in any way.

### EXAMPLES

### Example 1

### Milling of a Contrast Agent

Sterile WIN 67722 Suspension 150 mg/mL (referred to herein as “Sterile N1177”, “N1177 Injectable Suspension” or “N1177 drug product”) is a parenteral iodinated x-ray contrast agent which has been utilized for indirect lymphography. The N1177 compound is described, for example, in U.S. Pat. Nos. 5,322,679, 5,466,440, 5,518,187, 5,580,579, and 5,718,388. N1177 has the empirical formula C₁₉H₂₃I₃N₂O₆ and has the chemical name 6-ethoxy-6-oxohexy-3,5-bis(acetylamino)-2,4,6-triiodobenzoate, an esterified derivative of the X-ray contrast agent diatriazoic acid. N1177 has a molecular weight of 756.1. N1177 can be produced by the condensation of ethyl 6-bromohexanoate with sodium diatrizoate in DMF followed by the precipitation of the product from DMSO and washing with ethanol. N1177 can be obtained from Sigma-Aldrich Fine Chemicals.

The concentration of iodine in N1177 Injectable Suspension is 76 mg/mL. N1177 Injectable Suspension is a white to off-white crystalline material containing 50.35% iodine (by weight), and has a low water solubility (<10 μg/mL).

N1177 was milled to the desired particle size distribution. The particle size during milling was monitored in a preliminary study by periodically measuring particle size using XDC and PCS instruments. The results are shown below:

| | Mean Size D_{V}* | Mean Size D_{N} ** | Mean Effective |
|---|---|---|---|
| Time | (XDC) | (XDC) | Diameter (PCS) |
| T = 0 (unmilled) | 424 nm | 173 nm | 236 nm |
| Milled Day 3 | 148 nm | 84 nm | 158 nm |
| Milled Day 4 | — | — | 151 nm |
| Milled Day 5 | 143 nm | 79 nm | 149 nm |
| Milled Day 6 | — | — | 148 nm |
| Milled Day 7 | 138 nm | 77 nm | 147 nm |

*D_{N} is the preferred “number-weighted” size which is comparable to sizes determined by electron microscopy.
**D_{V} is “volume-weighted”.
Note that the PCS measurements are different than the XDC measurements as both techniques work differently. XDC measurements are considered more accurate, although PCS measurements are faster to produce.

The final formulation of N1177 Injectable Suspension for this example is as set forth below:

N1177 Formulation

| | MW | Molar Conc. | Mass Conc. |
|---|---|---|---|
| Component | (g/mole) | (M) | (mg/ml) |
| N1177 | 756.12 | 0.198 | 150 |
| Polyethylene glycol 1450 NF | ~15,000 | 0.01 | 150 |
| Poloxamer 338 | ~14,760 | 0.002 | 30 |
| Tromethamine-sufficient | 121.14 | 2.97 | 0.36 |
| to buffer to neutral pH | | | |

Relevant Formulation Specifications:
pH ~7.4

The polymeric excipients, poloxamer 338 and polyethylene glycol 1450, serve as particle stabilizers and are also intended to retard the rate of plasma clearance of particles by the reticuloendothelial system (RES) after intravascular administration. Poloxamer 338 is purchased from BASF®, and is purified by diafiltration as a part of the manufacturing process to reduce the level of low-molecular weight polymer.

### Example 3

### Detection of Macrophages in Atherosclerotic Plaques of Rabbits with N1177-iv-Enhanced Computed Tomography

Aortic atherosclerosis is induced in the male New Zealand White rabbits at a mean of 4 months and a mean weight of 3.3 kg. This is accomplished by 1) feeding the rabbits a high cholesterol diet for four months and by performing a double balloon denudation injury to the aorta. The same five rabbits are studied at three different doses of N1177: 125 mg/kg (dose 1), 250 mg/kg (dose 2, 1 week later), and 500 mg/kg (dose 3, 1 week after second dose).

Before imaging the animals are put under anesthesia by placing an intravenious access in the marginal vein of the ear with a 21-gauge line. Animals are kept in the same posture during all CT scans by placing them in a body-fitting thermosetting plastic holder. An initial localizer confirms the adequate position of the animal.

### Image Acquisition and Analysis

All animals are imaged by computed tomography angiography many times both before and after receiving a dose of contrast agent. Pre and post-N1177 image time points will be acquired for up to two hours. A scanner such as a 64-slice, multidetector-row computed tomography scanner may be used to acquire the imaging data. One possible instrument is the Sensation 64 by Siemens Medical Solutions, Forchheim, Germany. CT images are reconstructed on a computer and stored. Although various computer workstations may be used, one example of an adequate workstation for image processing is the Leonardo by Siemens Medical Solutions.

For each dose of N1177, and optimal imaging time point is determined. Atherosclerotic plaques are identified on axial images acquired during injection of N1177 as described above. For signal quantification, the density in the images is measured every 5 mm using appropriate image analysis software on 0.4 mm thick axial reconstructions by drawing a region of interest (ROI) in identified atherosclerotic plaque for each animal at each dosage level. An example of image analysis software which may be used is TeraRecon software (TeraRecon Inc., San Mateo, Calif., USA). The mean density of atherosclerotic plaque is expressed in Hounsfield units, and the experimental results are expressed in the enhancement in Hounsfield units for each atherosclerotic plaque between the images acquired before and after N1177 injection. All measurements are performed by two or more independent operators blinded to injection status, and the average of the measurements are used for the final analysis.

The enhancement measurements for atherosclerotic plaques are analyzed at each dosage level using a Student paired t-test to compare the signal intensity in the aortic wall before and after injection of N1177. The resulting probabilities are two-sided, expressed as measurement mean±standard deviation, with p-values <0.05 considered statistically significant. Standard statistical analysis software is used to perform the analysis. In this case, SPSS 12.0 is used, although software with similar capabilities may be used (e.g., MATLAB, R, SAS, etc).

### Histology

Rabbit plasma samples are also taken during the study to determine the drug exposure in the animals. This helps in assessing whether any additional toxicological studies are necessary. After imaging, the rabbits are euthanized by intravenous injection of sodium pentobarbital (120 mg/kg). A bolus of heparin is injected before euthanasia to prevent clot formation. Aortas are excised, fixed with formalin, and serial sections of the aorta are cut at 5-mm intervals. Coregistration, or proper alignment of the samples, is performed by utilizing the position of the renal arteries and iliac bifurcation. Selected aortic specimens are embedded in paraffin, and a 5 μm-thick sections are cut and stained with hematoxylin-eosin or with Massons's trichrome elastin. Immunostaining is performed with monoclonal antibodies against RAM-11, a marker of rabbit macrophage cytoplasm. Immunostaining images may be analyzed with any appropriate software. In this example, the images are analyzed with Image Pro Plus (Media Cybernetics). Macrophage rich areas (RAM-11 positive) are quantified on each specimen by computerized planimetry. Measurements of the luminal area, as well as the two areas bounded by internal and external elastic luminae, serve to computer intimal and medial areas and the intima/media ratio.

### Example 4

### Biodistribution and pKa of Large (GLP) and Small (FID) Particle Formulations in NZW Rabbits

The experiments of this example evaluate the pharmacokinetics and biodistribution of the large and small N1177 formulations following bolus injection of 6-ml of either the large (150 mg I/ml) or small (160 mg I/ml) formulations in NWZ rabbits.

Materials and Methods: The large (GLP-batch N1177-2002001-A, 150 mg I/ml; referred to as Compound A) and small (batch FID 2470, 160 mg I/ml; referred to as Compound B) were obtained from Nanoscan. The hydrated particle size of each batch was determined using dynamic laser light scattering methods.

Two NZW rabbits (3.3-3.8 Kg, number 07-467 (Compound A) and 07-458(Compound B)) were used in the current study. The rabbits were imaged using CT imaging. For both rabbits, pre CT scans were obtained followed by CT imaging during bolus phase of injection, 2 minutes post injection, and 2 hours post injection. Six-ml of each formulation was administered via bolus injection into the central ear vein at a rate of 0.6 ml/s. The signal in the liver and aorta was evaluated as a function of time post injection using the established protocols.

The pharmacokinetics were determined by drawing blood over a 5 minute to 15 hour time interval post injection (n=6 time points). Blood was collected into EDTA blood collection tubes via a catheter placed in the ear artery. All blood samples were sent to ICP-MS for determination of iodine content as function of time post injection. The blood half-lives were determined based on the resultant iodine (mg/mL) vs. time (pd.) curves. From these curves, kinetic parameters were obtained using standard non-compartmental biexponential pharmacokinetic analysis 24 hours after injection, the rabbits were sacrificed via overdose of ketamine/xylazine. The rabbits were then perfused with saline and the following organs excised, cleaned, and weighed: heart, liver, lung, spleen, kidney, and aorta. The organs were used for determination of iodine content. The percent-injected dose (% ID) was determined based upon the amount of iodine present in the tissue (mg) and the total amount of iodine administered (900 mg I for Compound A and 900 mg I for Compound B).

Results and Discussions: The hydrated particle size obtained for the two formulations is shown in Table 1. These results are consistent with previous studies that show the FID batch is significantly smaller than the original GLP batch.

TABLE 1
Size of large (Compound A) and small (Compound B) formulations

| Formulation | z-ave ± sigma | <d>v ± sd | <d>n ± sd | <d>I ± sd |
|---|---|---|---|---|
| GLP | 269.3 ± 0.223 | 296 ± 49, 83% | 269 ± 75, 100% | 328 ± 129, 100% |
| Compound A | | 524 ± 98, 16% | | |
| FID | 128.5 ± 0.379 | 30.6 ± 8, 50% | 27 ± 8, 99% | 35 ± 8, 6% |
| Compound B | | 106 ± 37, 14% | | 189 ± 94, 91% |

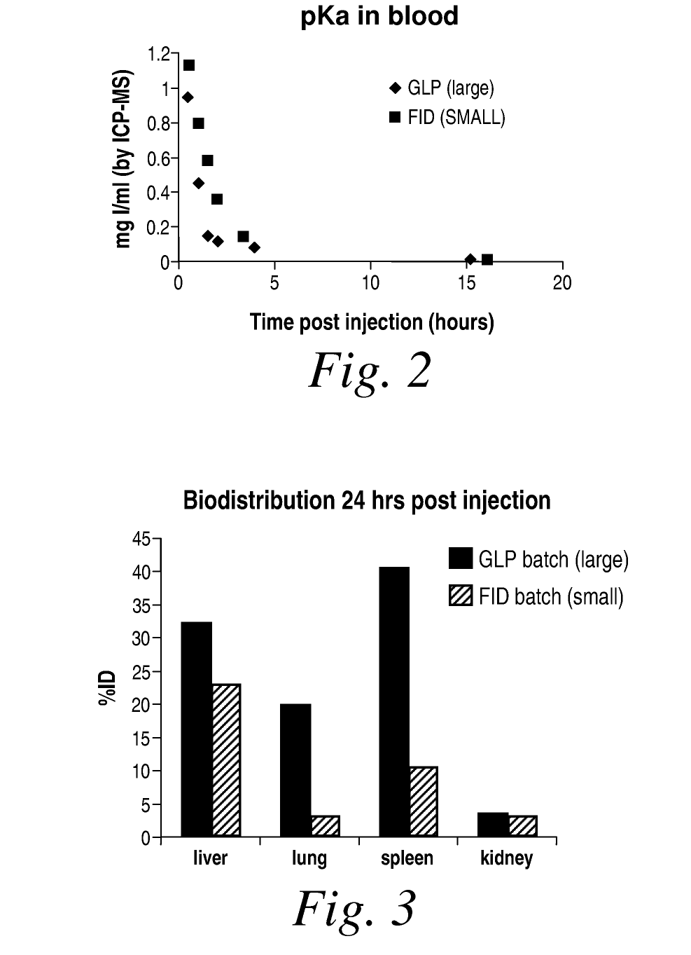

Due to movement of the rabbits between scans it was not possible to accurately access the CT signal attenuation (as Housenfeld Units; HU). However, on slices that could be matched it appears the CT signal may be increasing between 25 HU and 31 HU (relative to pre scans) following administration of the Compound B batch. Liver enhanced 14.6±5.5 HU 2 hours after administration of 6-ml of the Compound B batch. FIG. 2 summarizes the pK results. From the curve shown, the blood half-lives were obtained using both mono-exponential and bio-exponential fitting, as shown in Table 2. Table 2: Pharmacokinetics of the large (Compound A) and small (Compound B) formulations. T_{1/2} is the blood half-life obtained in rabbits after administration of a 6 mL bolus.

| | T_{1/2} (hrs) | T_{1/2} (hrs) Bi-exponential fit | | |
|---|---|---|---|---|
| Formulation | Mono-fit | T_{1/2} a (hrs) | T_{1/2} b (hrs) | Error |
| GLP | 0.48 | 1.8 | 0.4 | 0.0064 |
| Compound A | | | | |
| FID | 0.97 | 1.46 | 1.12 | 0.0017 |
| Compound B | | | | |

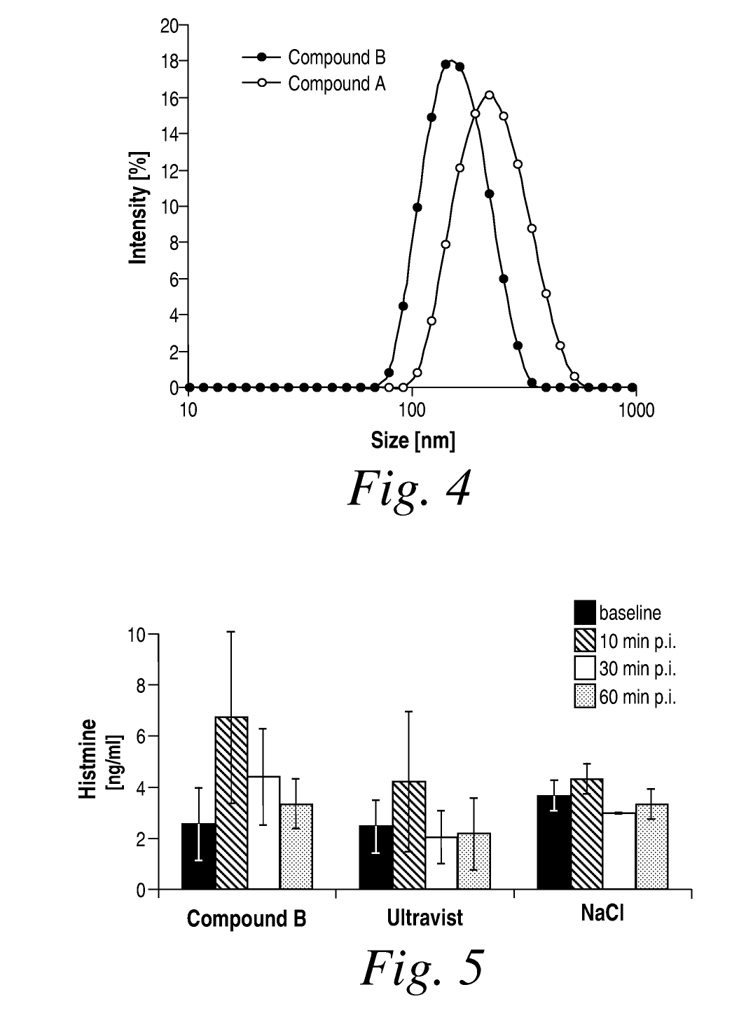

FIG. 3 shows % ID found in the liver, spleen, lung, and kidney obtained for the two formulations 24 hours after the administration of a 6 mL bolus. The results suggest that the large A batch has greater RES uptake (as observed by significant increases in the % ID of liver and spleen), relative to the smaller Compound B batch. Less than 4% of the injected dose was found in the kidneys 24 hours post injection for both formulations tested. No significant iodine was present in the heart. In the aorta 0.039% and 0.044% of the injected dose was obtained following administration of the large Compound A and small Compound B batches, respectively.

The pK and biodistribution studies strongly suggest that the small B formulation has a slower blood clearance and limited RES uptake, relative to the larger A batch. These findings correlate well with other studies that show iron oxide particle size greatly affects both the pK and biodistribution in rabbits. These results also suggest that the spleen is a critical elimination pathway for the large Compound A particles.

Conclusion: The results of this study strongly suggest that a reduction in particle size may increase the CT signal attenuation in the aorta, reduce the blood clearance (increase blood half-life) and limit RES uptake of the iodinated particles.

### Example 5

### Particle Size Measurements

Dynamic light scattering, also known as quasi elastic laser light scattering measures fluctuations of the scattered light intensity with time and is a technique which can be used to determine the hydrodynamic size of small particles in solution, as described above. By means of an autocorrelation of the intensity trace recorded during the experiment, the diffusion coefficient and, consequently, the hydrodynamic size of the particles can be obtained.

All measured samples were diluted 1:200 in water. The specified values for hydrodynamic size and polydispersity index were based on 5 individual measurements performed in triplicate and were calculated using the mode CONTIN.

In addition to the standard method DLS, the particle size was determined using a novel method for nanoparticle characterization—the Asymmetrical flow field-flow-fractionation (AF4). The benefit of the used AF4 is that the method is able to separate nanoparticle mixtures prior to DLS analysis (e.g., PCS). The AF4 experiments were performed with equipment purchased from Postnova Analytics GmbH (Landsberg, Germany).

The following settings were applied: injection time: 1 min; injection flow: 0.2 mL/min; tip flow rate: 0.70 mL/min; cross flow rate: 0.20 mL/min; detector flow rate: 0.50 mL/min. The aqueous solvent, a 0,2% FL70® detergent solution, was filtered through a 0.1 μm VacuCap® 90 filter unit (Pall Corporation, Germany) prior to use.

The channel length was 27.5 cm and the channel thickness was adjusted using a 350 μm spacer. The utilized ultrafiltration membrane was a regenerated cellulose acetate membrane having a 10 kDa cut-off (Postnova Analytics, Germany).

### Tested Compounds

Compound A: NanoCrystal™ Colloidal, Dispersion 150 mg/mL with 75 mg I/mL ZK: 6043014, Lot: GLP-N1177-20020001-A; Manufactured: 14. June 2002 (first batch)

Compound B: Nanoscan Imaging LLC, Dispersion 150 mg/mL; CT Contrast Agent, with 75 mg I/mL ZK: 6043014, Lot: 46-59, Mfg date: Feb. 10, 2007 (second batch)

### Results

TABLE 3
Size determination for compound A (Zetasizer Nano)

| Test | z- | Polydisperitäts- |
|---|---|---|
| sample | Average | index |
| 1 | 219 | 0.135 |
| 2 | 219 | 0.115 |
| 3 | 220 | 0.128 |

Diameter between 100 nm to 600 nm

### Method AF4:

Injection flow: 0.2 mL/min
Injection time: 2 min
Transion time: 2 min
Cross fow:60 min 0.2 mL/min
Detector flow:0.50 mL/min

### Spacer: 350 μm

By using the AF4 method a hydrodynamic diameter from 140 to 1050 nm was determined.
Average diameter for compound A:229.6 nm

TABLE 4
Size determination for compound B (Zetasizer Nano)

| Test | z- | Polydisperitäts- |
|---|---|---|
| sample | Average | index |
| 1 | 149 | 0.087 |
| 2 | 152 | 0.091 |
| 3 | 157 | 0.077 |

Diameter between 80 nm to 350 nm

### Method AF4:

Injection flow: 0.2 mL/min
Injection time: 2 min
Transion time: 2 min
Cross fow:60 min 0.2 mL/min
Detector flow:0.50 mL/min

### Spacer: 350 μm

By using the AF4 method a hydrodynamic diameter from 110 to 500 nm was determined. The average diameter for compound B:140 nm

### Conclusion

The first investigated batch (compound A, N1177-20020001-A) contained particle sizes from 100 to 600 nm with a hydrodynamic diameter between 140 to 1050 nm. The nano particles in dispersion of compound A showed a average particle size of 230 nm.

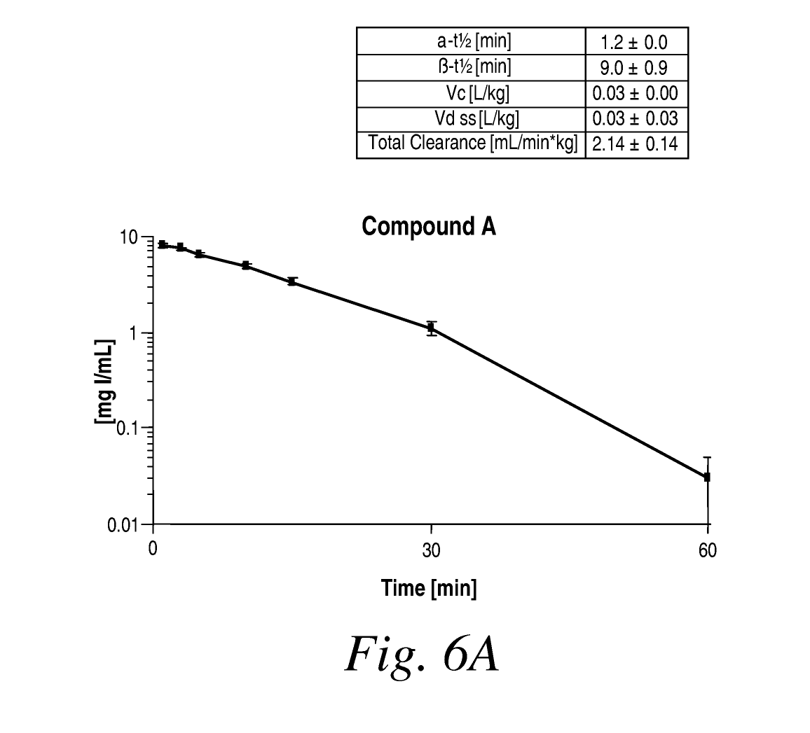

The second formulation (compound B, Lot: 46-59) is significantly smaller (particle sizes from 80 to 350 nm with a hydrodynamic diameter between 110 to 500 nm) and the size distribution is closer. The nano particles dispersion compound B showed a average particle size of 140 nm. See FIG. 4 for the distribution of particle sizes for compound A and B.

### Example 6

### Influence on Erythrocyte Morphology

A well-known adverse effect of some contrast agents is a decrease of the deformability of red blood cells, which results in alterations of their morphology and subsequent loss of their functionality.

### Materials and Methods

Effects on red blood cells can be monitored in vitro, by exposing human blood to contrast agents, monitoring the appearance of deformed erythrocytes and deriving a “damage index” (D.I.) immediately after exposure, 1 and 2 hours after incubation. Parameters: Morphological changes of blood erxthrocytes;

Range 0-5; value in % of maximal damage index (DI); (Spherocytes II and Haemolysis combined in DI 5=100%)

### Tested Compounds: Compound B

Incubated contrast media concentrations: 1, 2, 5 and 10 mg Iodine/mL blood
Reference substance: Ultravist 370, Ch.: 525096 370 mg I/mL
Contrast media was diluted with 0.9% NaCl-solution)

### Results

TABLE 5

| | | Conc. | Final | Damage index after | | |
|---|---|---|---|---|---|---|
| | | Solution | Conc. | incubation | | |
| sample | Charge | (mg I/mL) | (mg I/mL) | 0 h | 1 h | 2 h |
| Compound B | Lot | 70 | 10 | 2.8 | 1.7 | 6.3 |
| | 46-59 | 35 | 5 | 1.3 | 2.1 | 4.3 |
| | | 7 | 1 | 0.4 | 0.8 | 4.3 |
| Ultravist | 525096 | 370 | 52.86 | 31.2 | 12.2 | 24.4 |
| 0.9% NaCl | 7263A163 | | | 2.5 | 0.8 | 0.8 |
| Blood | | | | 0.0 | 1.3 | 0.4 |
| control | | | | | | |

In all concentrations, the damage index after compound B incubation are below the reference ULTRAVIST. Compound B has almost no damaging effects on the morphology of erythrocytes in this in vitro model.

### Example 7

### Histamine Release

These experiments examine the suitability of a nano particle formulation as contrast agent for CT investigations

### Materials and Methods

The release of histamine was investigated after single i.v. injection of 300 mg I/kg bw. A control group received 1 ml/kg of physiological saline (0.9% NaCl). Blood samples (approximately 1 mL) were taken via catheter from the carotid artery (using iced EDTA-Monovette® tubes) before dosing and at 10, 30, and 60 minutes after i.v. injection. After centrifugation (1500 g, 40° C., 20 minutes) for separation of the plasma, the samples were frozen at −80° C. until evaluation of histamine. Histamine was measured in the plasma samples using an ELISA system (RE 59221; IBL Immuno Biological Laboratories, Hamburg, Germany).

### Compound B: 72 mg I/mL

### Reference: Ultravist (300 mg I/mL)

Species: rats (Wistar Han), male (n=3)+female (n=3) Compounds; male (n=2)+female (n=2) Reference, 209-248 g
Application: i.v.—bolus

### Dose: 300 mg I/kg

Tested compounds: Compound B

TABLE 6

| | Histamine [ng/ml] | | | |
|---|---|---|---|---|
| Compound | baseline | 10 min p.i. | 30 min p.i. | 60 min p.i. |
| Compound B | 2.5 ± 1.4 | 6.7 ± 3.4 | 4.4 ± 1.9 | 3.3 ± 1.0 |
| Ultravist | 2.4 ± 1.0 | 4.2 ± 2.7 | 2.0 ± 1.0 | 2.2 ± 1.4 |
| 0.9% NaCl (KM | 3.7 ± 0.6 | 4.3 ± 0.6 | 3.0 ± 0.0 | 3.3 ± 0.6 |
| 04245) | | | | |

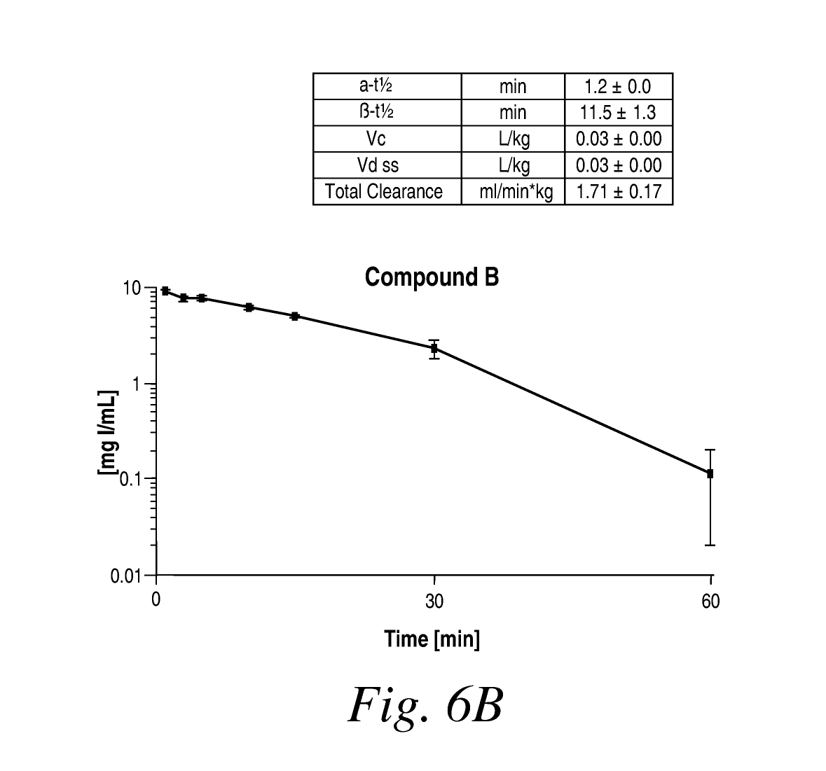

None of the probes, including the compound B, the reference (Ultravist), and saline control did induce a large or persistent increased histamine release in the tested rats. All values were in a normal physiological range (literature data). After application the animals didn't show side effects. FIG. 5 shows plasma histamine levels after intravenous injection of compound B in comparison to Ultravist and NaCl in rats. There was a no release of histamine after injection of nano-particle formulation (compound B) at a dose of 300 mg I/kg bw in the conscious rat model.

### Example 8

### Acute Toxicity Study in Mice—LD₅₀

The LD₅₀ is usually expressed as the mass of substance administered per unit mass of test subject, such as grams of substance per kilogram of body mass.

### Materials and Methods

Animals: SPF-mice, 18-22 g, f:m=50:50 (n=6)

### Concentration of CM-Solution: 75 mg I/mL

Injection speed: 2 mL/Min i.v.
Basic criteria: exitus letalis
Time of observation: 7 days
Tested compounds: Compound A and Compound B

### Results

LD50 acute toxicity of nano-crystal suspension compound A app. 3.75 g Iodine/kg
LD50 acute toxicity of nano-crystal suspension compound B app. 5.5 g Iodine/kg

### Conclusion

The LD₅₀ of the smaller suspension, compound B, was higher compared to compound A, but significantly lower compared to established marketed Iodine containing contrast media (Ultravist >12 g Iodine/kg bw)

### Example 9

### Plasma Kinetic Study after I.V. Injection in Rats

The blood/plasma elimination kinetics were examined in rats. Pre and up to 24 h after i.v. injection of the compounds blood samples were taken and the concentrations was quantified via RFA/ICP. Pharmacokinetic parameters (Vss, Cltot, elimination half-life, etc.) were calculated using PC based software package (Win NonLin).

### Materials and Methods

Animals: Han Wistar rats, male, 292-307 g, n=3 per investigated compound

### Conc.: 75 mg I/mL

Dosage: 250 mg I/kg iv bolus
Blood samples were taken after: 1, 3, 5, 10, 15, 30, 60, 90, 120, 240, 360 and 1440 minutes p.i.

### Tested Compounds: Compound A and Compound B

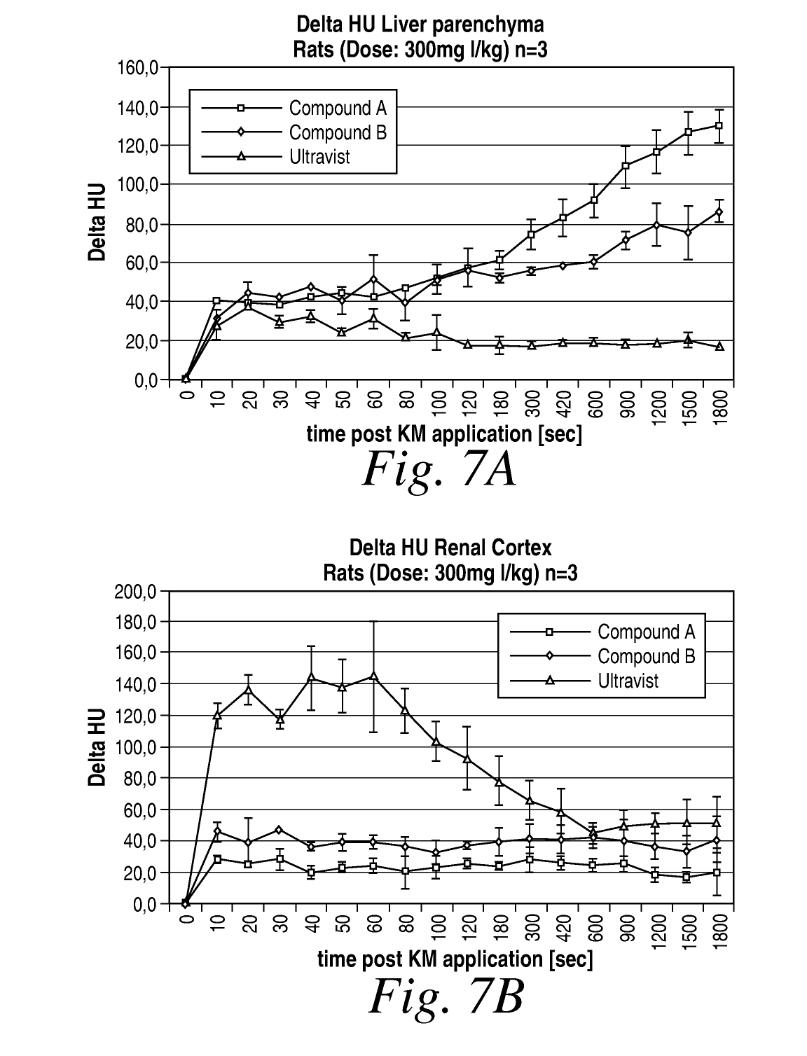

The smaller particles (compound B) were more slowly eliminated (beta half time 11.5 versus 9.0) and showed a longer blood enhancement compared to the larger particles (see FIG. 6 for plasmakinetics of compounds A and B). The investigated nano-crystal formulations showed excellent blood pool characteristics compared to currently marketed extracellular x-ray contrast media like Ultravist.

### Example 10

### Biodistribution and Elimination Study after I.V. Injection in Rats

To evaluate the overall elimination of the compounds from body, rats were used. After injection of the compounds the animals were placed in metabolic cages, and urine and feces were sampled up to 7 days post injection (d.p.i.) for quantification (RFA, ICP) of eliminated fractions. At the end of the observation period (7 d.p.i.) the animals were sacrificed and the overall (body; organs and carcass) retention of the compounds was examined (RFA, ICP).

### Materials and Methods

Species: rats, female, 96-97 g, n=3 per investigated compound
Application: i.v.—bolus

### Dose: 300 mg I/kg

Elimination: urine and feces daily 1st to 7th day, additionally (urine only) 1 h, 3 h, 6 h
p.i. Biodistribution: 7 days p.i.: liver, kidneys, stomach/intestine (empty) and carcass
Evaluation: amount Iodine in the samples using RFA, evaluation in mg I/mL, μmol I/L and % Dose.

### Tested Compounds: Compound A and Compound B

### Results

TABLE 7
Biodistribution and Elimination 7 days after i.v. injection in rats of
compound A

| | | | | Amount of I cumulative | |
|---|---|---|---|---|---|
| | Amount of I in | | | in [% Dose] | |
| Organ | μmol/l | % Dose | Time | Urine | Feces |
| liver | 755 ± 289 | 12.4 ± 3.2 | 1 h | 4.8 ± 0.3 | — |
| kidneys | b.d.l. | b.d.l. | 3 h | 5.6 ± 0.3 | — |
| stomach/ | b.d.l. | b.d.l. | 6 h | 7.0 ± 0.3 | — |
| intestine | | | | | |
| Carcass | 28 ± 4 | 3.9 ± 0.5 | 1 d | 11.5 ± 0.9 | 36.8 ± 11.2 |
| Total | — | 16.4 ± 3.7 | 2 d | 14.7 ± 2.6 | 43.2 ± 6.0 |
| | | | 3 d | 16.5 ± 2.9 | 46.1 ± 6.4 |
| | | | 4 d | 18.1 ± 3.3 | 48.4 ± 6.3 |
| | | | 5 d | 19.5 ± 4.0 | 50.2 ± 6.7 |
| | | | 6 d | 20.4 ± 4.5 | 51.4 ± 6.4 |
| | | | 7 d | 21.4 ± 5.1 | 52.8 ± 6.2 |
| | | | Total | 21.4 ± 5.1 | 52.8 ± 6.2 |

Total recovery 93.8 ± 2.4

TABLE 8
Biodistribution and Elimination 7 days after i.v. injection in rats of
compound B

| | | | | Amount of | |
|---|---|---|---|---|---|
| | | | | I cumulative | |
| | Amount of I in | | | in [% Dose] | |
| Organ | μmol/l | % Dose | Time | Urine | Feces |
| liver | 425 ± 50 | 5.7 ± 0.6 | 1 h | 12.4 ± 0.8 | — |
| kidneys | b.d.l. | b.d.l. | 3 h | 15.1 ± 2.3 | — |
| stomach/intestine | b.d.l. | b.d.l. | 6 h | 16.5 ± 2.9 | — |
| Carcass | b.d.l. | b.d.l. | 1 d | 22.9 ± 3.2 | 45.7 ± 5.1 |
| Total | — | 5.7 ± 0.6 | 2 d | 25.3 ± 5.5 | 52.4 ± 2.6 |
| | | | 3 d | 25.3 ± 5.5 | 56.3 ± 1.7 |
| | | | 4 d | 25.3 ± 5.5 | 58.5 ± 1.5 |
| | | | 5 d | 25.3 ± 5.5 | 58.5 ± 1.5 |
| | | | 6 d | 25.3 ± 5.5 | 58.5 ± 1.5 |
| | | | 7 d | 25.3 ± 5.5 | 58.5 ± 1.5 |
| | | | Total | 25.3 ± 5.5 | 58.5 ± 1.5 |

Total recovery 91.3 ± 1.7

Both nano-crystal formulations were eliminated approximately two-thirds via the liver (53% vs. 59%) and one third renally (22% vs. 26%). In this experiment the recovery was approximately 92% of the total dose.

After application of the larger particle formulation (compound-A) on day 7 p.i. a higher iodine concentration uptake in liver (12.4% dose) and carcass (3.9% dose) could be observed. In contrary, no iodine uptake was measured in carcass 7 days after application of the smaller particle formulation (compound B) and the uptake in liver was lower (5.7% dose). The size of the particles has a significant influence on the Biodistribution and Elimination.

### Example 11

### Contrast Media Kinetic Study: Investigation in Computed Tomography

A comparative study in computed tomography was performed between a established CT-contrast media (Ultravist) and the nano-crystal formulation. The goal was to investigate the suitability of for the use of nanocrystal formulations in modern CT.

The CT—number (Hounsfield Units (HU)) were determined in respective regions of interest (ROI) in kidney, aorta, vena cava and liver.

### Materials and Methods

### Hardware: CT Siemens Volume Zoom

Imaging parameters: Sequence, table speed=0; tube voltage: 80 kV; tube current 100 mAs, scan time: 1 s; reconstruction kernel: B40, 4×2.5 mm; intervals: Δt=3 s (0-300 s); Δt=10 s (300-1800 s)
Contrast Media Application and animals:
Han Wistar rats, 300 mgI/kgBW via bolus-application about tail vein
Group 1: Ultravist 300 (n=3)
Group 2: Compound A (n=3)
Group 2: Compound B (n=3)
Tested compounds: Compound A and Compound B

### Results

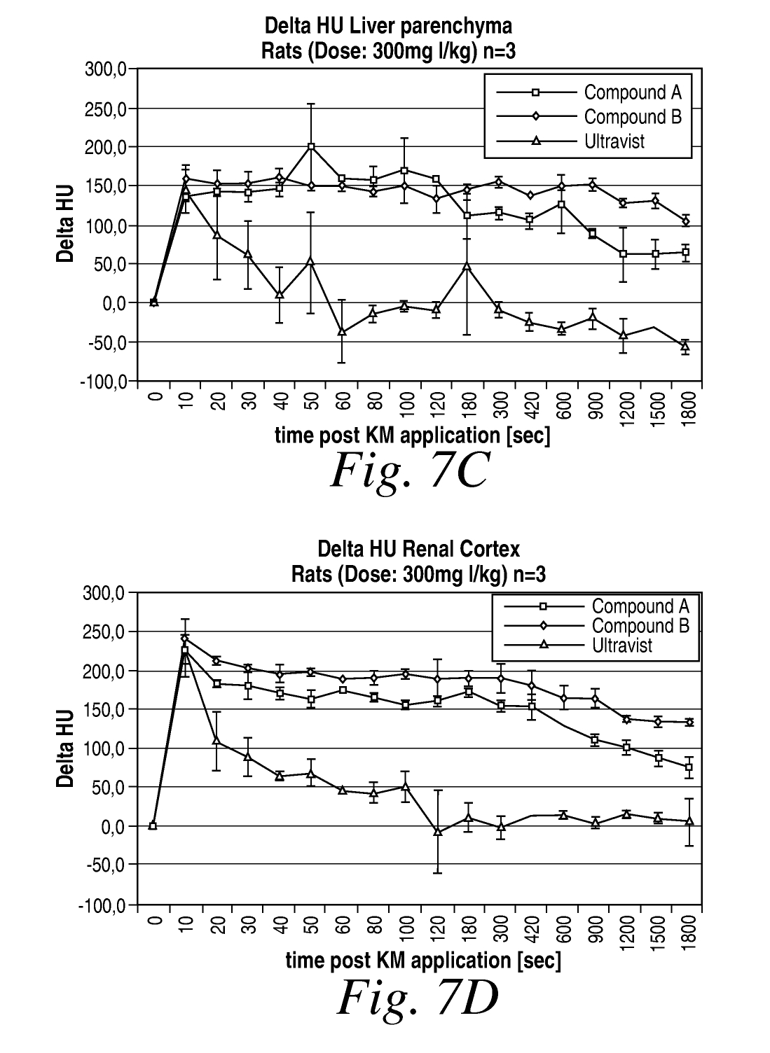

The nanocrystal formulations showed different properties regarding temporal course in liver, kidney and vessel enhancement comparing it with well-established x-ray contrast agent like Ultravist. By using the same total injected dose, the HU values especially in liver, aorta and vena cava were significantly higher (See FIG. 7 for HU values in the liver, renal cortex, aorta, and vena cava).

From an imaging perspective the nano-crystal formulations showed a prolonged vessel opacification, as would be expected of a dedicated blood pool agent. The uptake in liver and the biliary elimination of the investigated compounds allows a visualization of liver parenchyma. These results generally demonstrate that the contrast agents of the invention are more effective for imaging the blood pool of the liver parenchyma, the aorta, and the vena cava. This further demonstrates the effectiveness of the contrast agents of the invention to better image blood vessels.

### Example 12

### Exploratory Study in a Tumor Bearing Rabbit: CT-Tumor Perfusion Imaging

The goal of the study was to explore the potential of different contrast media to visualize tumor perfusion.

### Materials and Methods

Animal model: rabbit VX2-tumor
CT: dynamic measurements (0-80 s) at 100 kV, 1 image/s
Tested compounds: Compound B; Ultravist 300;
dose: 300 mgI/kg b.w.

### Results

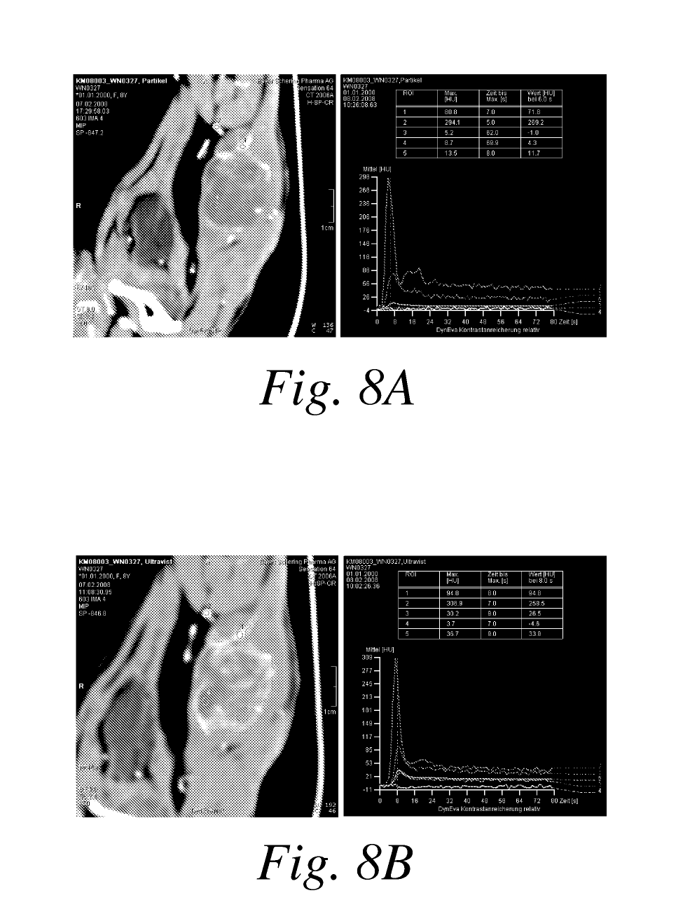

In this experiment the influence of the particle size on the tumor perfusion was observed. Ultravist as a small molecule caused an intra- and extravasal perfusion. In contrary compound B showed only a intravasal tumor perfusion (See FIG. 8). For both compounds, the HU values for blood vessel enhancement were comparable (see Table 9).

TABLE 9
Measurement of the HU values

| ROI | | Ultravist [HU] | N1177 [HU] |
|---|---|---|---|
| 1 | Tumor vessel | 95 | 81 |
| 2 | Vessel | 294 | 307 |
| 3 | Tumor vital tissue | 30 | 5 |
| 4 | Tumor necrotic tissue | 4 | 9 |
| 5 | Whole tumor | 37 | 13 |

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A composition comprising a crystalline iodinated nanoparticle contrast agent having a mean particle size of about 100 nanometers to about 150 nanometers.

2. A composition comprising a crystalline iodinated nanoparticle contrast agent having a particle size distribution of between about 80 and about 350 nanometers in diameter as measured by asymmetrical flow field fractionation.

3. A composition comprising a crystalline iodinated nanoparticle contrast agent having a particle size distribution of between about 20 and about 120 nanometers in diameter as measured by photon correlation spectroscopy (PCS).

4. A composition comprising a crystalline iodinated nanoparticle contrast agent having a particle size distribution in which 100% of particles are less than about 200 nanometers as measured by X-ray disc centrifuge sedimentometry (XDC).

5. The composition of any one of claims 1-4, wherein said contrast agent is an ester of diatrizoic acid.

6. The composition of claim 5, wherein said contrast agent comprises iodine.

7. The composition of claim 6, wherein the contrast agent is 6-ethoxy-6-oxohexy-3,5-bis(acetylamino)-2,4,6-triiodobenzoate

8. An in vivo method for obtaining an image of accumulated macrophages in a blood vessel of a subject comprising:
a) administering an effective amount of a composition comprising a nanoparticulate contrast agent having mean diameter of less than or equal to about 150 nanometers to the subject intravenously; and
b) detecting the contrast agent.

9. An in vivo method for obtaining an image of plaque accumulation in a blood vessel of a subject comprising:
a) administering an effective amount of a composition comprising a nanoparticulate contrast agent having a mean diameter of about 150 nanometers or less the subject intravenously; and
b) waiting a time sufficient after administration of the contrast agent to allow the contrast agent to be taken up by macrophages in vascular plaque that may be present in the subject; and
c) detecting the contrast agent taken up by the macrophages thereby obtaining an image of vascular plaque that may be present in the subject.

10. An in vivo method for predicting risk of vascular disease by obtaining and evaluating an image of accumulated macrophages within a blood vessel of a subject comprising:
a) administering to the subject an effective amount of a composition comprising a nanoparticulate contrast agent having a mean diameter of about 150 nanometers or less;
b) waiting a time sufficient after administration of the contrast agent to allow the contrast agent to be taken up by macrophages in vascular plaque that may be present in the subject; and
c) detecting the contrast agent taken up by the macrophages thereby obtaining an image of accumulated macrophages that may be present in the subject.
d) predicting risk of vascular disease in the subject based on the image formed.

11. The method of claim 10, wherein the prediction is made based on a quantitative measure of the accumulation of the contrast agent in the macrophages in the vessel wall of the subject.

12. The method of claim 10, wherein said vascular disease is selected from the group consisting of atherosclerosis, coronary artery disease (CAD), myocardial infarction (MI), ischemia, stroke, peripheral vascular diseases, and venous thromboembolism.

13. A method for diagnosing atherosclerosis in a human subject, comprising
a) examining an image for the presence or absence of vascular plaque, wherein the image is obtained by:
i. administering to a human subject at risk for developing vascular plaque an effective amount of a composition comprising the nanoparticulate contrast agent 6-ethoxy-6-oxohexy-3,5-bis(acetylamino)-2,4,6-triiodobenzoate having a mean diameter of about 150 nanometers or less;
ii. waiting a time sufficient after administration of the contrast agent to allow the contrast agent to be taken up by macrophages and for the amount of the contrast agent in the lumen of the vessel to be imaged to be reduced to an amount which allows macrophages in vascular plaque that may be present in the human subject to be visualized; and
iii. detecting the contrast agent taken up by the macrophages, and
b) concluding whether vulnerable plaque is present in the image, wherein the presence of vascular plaque is indicative of atherosclerosis, to thereby diagnose atherosclerosis in the human subject.

14. An in vivo method for obtaining an image of vulnerable vascular plaque that may be present in a subject at risk for developing vascular plaque, comprising
a) administering to a subject at risk for developing vascular plaque or known to have a vascular plaque an effective amount of a composition comprising a nanoparticulate contrast agent having a mean diameter of about 150 nanometers or less;
b) waiting a time sufficient after administration of the contrast agent to allow the contrast agent to be taken up by macrophages in vulnerable vascular plaque that may be present in the subject; and
c) constructing an image from data obtained by detecting the contrast agent taken up by the macrophages to thereby obtaining an image of vulnerable vascular plaque that may be present in the subject.

15. An in vivo method for obtaining an image of a vascular blood pool in a subject comprising:
a) administering an effective amount of a composition comprising a nanoparticulate contrast agent having a mean diameter of about 150 nanometers or less to the subject intravenously; and
b) detecting the contrast agent present in a blood vessel of the subject to thereby obtain an image of a vascular blood pool in a subject.

16. The method of claim 15 wherein the vascular blood pool is chosen from the group consisting of a liver blood pool, a pancreatic blood pool, a lung blood pool, a cardiac blood pool, a splenic blood pool, and a brain blood pool.

17. The method of claim 16 wherein the vascular blood pool is a cardiac blood pool.

18. The method of claim 16 wherein the vascular blood pool is a splenic blood pool.

19. The method of claim 16 wherein the vascular blood pool is a pancreatic blood pool.

20. The method of claim 16 wherein the vascular blood pool is a lung blood pool.

21. The method of claim 16 wherein the vascular blood pool is a brain blood pool.

22. An in vivo method for obtaining an image of phagocytic cells in the brain of a subject comprising:
a) administering an effective amount of a composition comprising a nanoparticulate contrast agent having a mean diameter of about 150 nanometers or less to the subject intravenously; and
b) waiting a time sufficient after administration of the contrast agent to allow the contrast agent to be taken up by phagocytic cells that may be present in the brain of the subject; and
c) detecting the contrast agent taken up by the phagocytic cells thereby obtaining an image of phagocytic cells that may be present in the brain of the subject.

23. The method of claim 22, wherein the phagocytic cells are associated with neural plaques.

24. An in vivo method for detecting the presence of a tumor that may be present at a site of interest in a subject comprising:
a) administering an effective amount of a composition comprising a nanoparticulate contrast agent having a mean diameter of about 150 nanometers or less to the subject intravenously such that it is present in the vasculature of the subject; and
b) detecting the contrast agent present in the vasculature at the site of interest,
to thereby obtain an image of the vasculature associated with a tumor that may be present at a site of interest in the subject.

25. The method of claim 24, wherein the image is evaluated for areas of increased formation of blood vessels or leakage of contrast agent from blood vessels.

26. An in vivo method for obtaining an image of phagocytic cells at a site of interest in a subject comprising:
a) administering an effective amount of a composition comprising a nanoparticulate contrast agent having a mean diameter of about 150 nanometers or less to the subject intravenously; and
b) waiting a time sufficient after administration of the contrast agent to allow the contrast agent to be taken up by phagocytic cells that may be present at the site of interest in the subject; and
c) detecting the contrast agent taken up by the phagocytic cells thereby obtaining an image of phagocytic cells that may be present at the site of interest in the subject.

27. The method of claim 26, wherein the phagocytic cells are present at the site of a tumor.

28. The method of any one of claim 8, 9, 10, or 13-29 wherein the composition a particle size distribution in which 100% of the contrast agent has a particle size of not more than 400 nanometers.

29. The method of any one of claim 8, 9, 10, or 13-27 wherein the contrast agent has a mean particle size of between about 100 and 150 nm.

30. The method of any one of claim 8, 9, 10 , or 13-27 wherein the contrast agent has a particle size distribution of between about 80 and about 350 nanometers in diameter as measured by asymmetrical flow field fractionation.

31. The method of any one of claim 8, 9, 10 , or 13-27 wherein the contrast agent has a particle size distribution of between about 20 and about 120 nanometers in diameter as measured by photon correlation spectroscopy (PCS).

32. The method of any one of claim 8, 9, 10 , or 13-27 wherein the contrast agent has a mean particle size of between about 100 nm and 150 nm and a particle size distribution of between about 80 and about 350 nanometers in diameter as measured by asymmetrical flow field fractionation or of between about 20 and about 120 nanometers in diameter as measured by photon correlation spectroscopy (PCS).

33. The method of any one of claim 8, 9, 10 , or 13-21 wherein the method of detecting is selected from the group consisting of: x-ray imaging, computed tomography (CT), computed tomography angiography (CTA), multi-detector CT (MDCT), electron beam (EBT), magnetic resonance imaging (MRI), magnetic resonance angiography (MRA), and positron emission tomography.
